SEQUENCE LISTING (25 sequences)

SEQ ID NO: 1 - DNA, 1274 bp, synthetic construct
  source 1..1274: mol_type = other DNA
tctagaccaccatgaagttgcctgttaggctgttggtgctgatgttctggattcctgcttccagcagtgacatccaaatgacccagagccccagtagcctgagtgccagcgtgggcgacagggtgaccatcacctgcagtgcaagcagcagcgtgagctacatgaactggtatcaacagaccccaggcaaggcgcccaaaaggtggatctacgacaccagcaagctggcgagcggtgtgcccagcaggttttccggtagtgggtcaggcaccgactacaccttcactatcagcagcctgcagcccgaggacatcgccacctattactgccagcaatggagcagcaaccccttcaccttcggacagggcacgaagctgcagatcacgaggggtggaggcggtagcggtgggggaggctcagggggcggaggcagtggcggtggcgggagcgggggtggtggttcaggaggaggtggcagtcaggtgcagctggtgcagagcggagggggtgtggtacagcccggcaggagcttgaggctgagctgcaaggccagcggctatacctttacgaggtacaccatgcactgggtgaggcaagcgcctggtaagggcctggagtggatcgggtacatcaaccctagcaggggctacactaactacaaccagaagttcaaagacagattcacgattagcacagataagagcaagtctaccgctttcctccagatggacagccttaggcctgaggacaccgccgtgtactactgcgcaaggtactacgacgaccactactgtcttgactattgggggcaggggactcccgtgaccgtgagcagcggtggaggaggtagtggaggaggcggaagtggtggtggaacatcgggaggcggaggttcaggaggtggaggctctcaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagcacttacttactacgattacgaatttgcttactggggccaagggactctggtcactgtctctgcagctagc

SEQ ID NO: 2 - protein, 421 aa, synthetic construct
MKLPVRLLVLMFWIPASSSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGGGGSGGGGSGGGGSGGGGSGGGGSGGGGSQVQLVQSGGGVVQPGRSLRLSCKASGYTFTRYTMHWVRQAPGKGLEWIGYINPSRGYTNYNQKFKDRFTISTDKSKSTAFLQMDSLRPEDTAVYYCARYYDDHYCLDYWGQGTPVTVSSGGGGSGGGGSGGGTSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAAS

SEQ ID NO: 3 - DNA, 737 bp, synthetic construct
  source 1..737: mol_type = other DNA
tctagagccgccaccatgaagttgcctgttaggctgttggtgctgatgttctggattcctgcttccagcagtgacatcttgctgacacaatctccagtcatcctgtctgtgagtccaggagaaagagtcagtttctcctgcagggccagtcagagcattggcacaaacatccactggtatcaacagagaacaaatggttctccaaggcttctcataaagtatgcttctgagtctatttctgggatcccttccaggtttagtggcagtggatcagggacagactttactcttagcatcaacagtgtggagtctgaagatattgcagattattactgtcaacaaaataataactggcctacaacgttcggtgcaggaacgaagctggaactcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccggatcc

SEQ ID NO: 4 - protein, 233 aa, synthetic construct
MKLPVRLLVLMFWIPASSSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, synthetic construct
  VARIANT 4: note = X = G or T
GGGXS

SEQ ID NO: 6 - protein, 5 aa, synthetic construct
GGGGS

SEQ ID NO: 7 - protein, 5 aa, synthetic construct
GGGTS

SEQ ID NO: 8 - protein, 25 aa, synthetic construct
  VARIANT 1..24: note = Each X is independently G or T
GGGXSGGGXSGGGXSGGGXSGGGXS

SEQ ID NO: 9 - protein, 25 aa, synthetic construct
GGGGSGGGGSGGGGSGGGGSGGGGS

SEQ ID NO: 10 - protein, 25 aa, synthetic construct
GGGGSGGGGSGGGTSGGGGSGGGGS

SEQ ID NO: 11 - protein, 30 aa, synthetic construct
  VARIANT 4..29: note = Each X is independently G or T
GGGXSGGGXSGGGXSGGGXSGGGXSGGGXS

SEQ ID NO: 12 - protein, 30 aa, synthetic construct
GGGGSGGGGSGGGGSGGGGSGGGGSGGGGS

SEQ ID NO: 13 - protein, 30 aa, synthetic construct
GGGGSGGGGSGGGTSGGGGSGGGGSGGGGS

SEQ ID NO: 14 - protein, 10 aa, synthetic construct
SASSSVSYMN

SEQ ID NO: 15 - protein, 7 aa, synthetic construct
DTSKLAS

SEQ ID NO: 16 - protein, 9 aa, synthetic construct
QQWSSNPFT

SEQ ID NO: 17 - protein, 13 aa, synthetic construct
KASGYTFTRYTMH

SEQ ID NO: 18 - protein, 17 aa, synthetic construct
YINPSRGYTNYNQKFKD

SEQ ID NO: 19 - protein, 12 aa, synthetic construct
YYDDHYCLDYWG

SEQ ID NO: 20 - protein, 10 aa, synthetic construct
GFSLTNYGVH

SEQ ID NO: 21 - protein, 16 aa, synthetic construct
VIWSGGNTDYNTPFTS

SEQ ID NO: 22 - protein, 11 aa, synthetic construct
ALTYYDYEFAY

SEQ ID NO: 23 - protein, 11 aa, synthetic construct
RASQSIGTNIH

SEQ ID NO: 24 - protein, 7 aa, synthetic construct
YASESIS

SEQ ID NO: 25 - protein, 9 aa, synthetic construct
QQNNNWPTT

(19) United States
(12) Patent Application Publication
(10) Pub. No.: US 2025/0319127 A1
(43) Pub. Date: Oct. 16, 2025

(54) BISPECIFIC ANTIBODIES

(71) Applicants: Manley Huang, Palo Alto, CA (US); University of Virginia Patent Foundation, Charlottesville, VA (US); TransTarget Inc., Sunnyvale, CA (US)

(72) Inventors: Lawrence G. Lum, Charlottesville, VA (US); Manley Huang, Palo Alto, CA (US); James Larrick, Sunnyvale, CA (US); Vikram Sharma, San Francisco, CA (US); Andrew Mendelsohn, Palo Alto, CA (US); Bo Yu, Mountain View, CA (US)

(21) Appl. No.: 18/705,955

(22) PCT Filed: Oct. 31, 2022
(86) PCT No.: PCT/US2022/079005

§ 371 Date: Apr. 29, 2024

Related U.S. Application Data
(60) Provisional application No. 63/273,805, filed on Oct. 29, 2021.

(51) Int. Cl. A61K 35/17; A61K 40/11; A61K 40/31; A61K 40/42; A61P 3/10; A61P 35/00; C07K 16/28; C07K 16/32

(52) U.S. Cl. CPC A61K 35/17; A61K 40/11; A61K 40/31; A61K 40/4204; A61K 40/4205; A61K 40/4224; A61P 3/10; A61P 35/00; C07K 16/2809; C07K 16/2863; C07K 16/2896; C07K 16/32; A61K 2239/13; A61K 2239/29; C07K 2317/31; C07K 2317/622

(57) ABSTRACT

Provided are tetravalent bispecific antibodies (T-BiAbs) that have a first binding moiety and a second binding moiety, wherein the first binding moiety is a single chain variable fragment (scFv) and the second, binding moiety-7 is a monoclonal antibody, and further wherein the variable light (Vi.) and variable heavy7 (VH) chains of the first binding moiety7 are directly linked as a. single chain to the second binding moiety at the N-terminus or the C-terminus of the light chain or the heavy chain sequence of the second binding moiety. In some embodiments, the first binding moiety binds to a CDS polypeptide and the second binding moiety-7 binds to a tumor-associated antigen. Also provided are T cells armed with the presently disclosed T-BiAbs and methods of using the same for treating tumor and/or cancers, treating diabetes, arming and isolating stem cells, and manufacturing medicaments for these purposes.

### CROSS REFERENCE TO RELATED APPLICATION

The presently disclosed subject matter claims the benefit of U.S. Provisional Patent Application Ser. No. 63/273,805, filed Oct. 29, 2021, the disclosure of which incorporated herein by reference in its entirety.

### REFERENCE TO SEQUENCE LISTING XML

The Sequence Listing XML associated with the instant disclosure has been electronically submitted to the United States Patent and Trademark Office via the Patent Center as a 25,236 byte UTF-8-encoded XML file created on Oct. 31, 2022 and entitled “3062_174_PCT.xml”. The Sequence Listing submitted via Patent Center is hereby incorporated by reference in its entirety.

A Substitute Sequence Listing XML has been electronically submitted to the United States Patent and Trademark Office via the Patent Center as a 26,651 byte UTF-8-encoded XML file created on Jan. 7, 2025 and entitled “Updated_3062_174_PCT.xml”. The Substitute Sequence Listing submitted via Patent Center is hereby incorporated by reference in its entirety.

### TECHNICAL FIELD

The presently disclosed subject matter relates generally to tetravalent bispecific antibodies (T-BiAb) in which the variable light (V_{L}) and heavy (V_{H}) chains of a first antibody are directly linked as a single chain variable fragment (scFv) to a second monoclonal antibody with a different binding specificity. In some embodiments, the scFv of the first antibody is attached to the N- or C-terminus of either the light chain or the heavy chain sequence of the second antibody.

### BACKGROUND

The advent of hybridoma and antibody engineering technologies to generate humanized monoclonal antibodies has led to the regulatory approval of over 70 antibodies or fragments worldwide. Subsequent technologies to engineer antibody variable regions with or without Fc effector functions have broadly expanded the range of clinical applications. In particular, widespread efforts have led to a broad variety of bispecific antibody variants with novel arrangements of V_{H} and V_{L} chain domains depending on the need for monovalent or bivalent specificities and associated effector functions. However, challenges remain regarding protein stability, expression levels, retention of antibody binding affinity and activity, and production of high quantities of intact proteins. The presently disclosed subject matter describes a general approach to the efficient production of tetravalent bispecific antibodies (T-BiAb) and compositions thereof.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to tetravalent bispecific antibodies (T-BiAbs) comprising a first binding moiety and a second binding moiety. In some embodiments, the first binding moiety is a single chain variable fragment (scFv) and the second binding moiety is a monoclonal antibody. In some embodiments, the variable light (V_{L}) and variable heavy (V_{H}) chains of the first binding moiety are directly linked as a single chain to the second binding moiety at the N-terminus or the C-terminus of the light chain or the heavy chain sequence of the second binding moiety. In some embodiments, the variable light (V_{L}) and variable heavy (V_{H}) chains of the first binding moiety are linked to each other as a single polypeptide chain via a peptide linker. In some embodiments, the peptide linker comprises, consists essentially of, or consists of the amino acid sequence GGGGS (SEQ ID NO: 6), optionally wherein the peptide linker comprises, consists essentially of, or consists of a concatemer of 3-6 copies of the amino acid sequence GGGGS (SEQ ID NO: 6). In some embodiments, at least one of the copies of the amino acid sequence GGGGS (SEQ ID NO: 6) includes an amino acid substitution to GGGTS (SEQ ID NO: 7), optionally wherein the peptide linker comprises, consists essentially of, or consists of the amino acid sequence GGGGSGGGGSGGGTSGGGGSGGGGS (SEQ ID NO: 10) or GGGGSGGGGSGGGTSGGGGSGGGGSGGGGS (SEQ ID NO: 13). In some embodiments, the variable light (V_{L}) and variable heavy (V_{H}) chains of the scFv are linked to each other in a configuration selected from the group consisting of V_{L}-(G4S)ₓ-V_{H} and V_{H}-(G4S)ₓ-V_{L}, wherein G4S is the amino acid sequence GGGGS (SEQ ID NO: 6) or a threonine-containing variant thereof and x is 3-6. In some embodiments, the scFv binds to a CD3 polypeptide, optionally wherein the scFv is an scFv of an OKT3 monoclonal antibody. In some embodiments, the scFv binds to a CD3 polypeptide and the second binding moiety binds to a tumor-associated antigen. In some embodiments, the tumor-associated antigen is a polypeptide selected from the group consisting of an ERBB family member polypeptide, optionally an epidermal growth factor receptor (EGFR/ERBB1) polypeptide, a HER2/ERBB2 polypeptide, a HER3/ERBB3 polypeptide, a HER4/ERBB4 polypeptide, a disialoganglioside 2 (GD2) polypeptide, a MAG-1 polypeptide, a CD19 polypeptide, a CD20 polypeptide, a CD22 polypeptide, a CD30 polypeptide, a CD33 polypeptide, a CD34 polypeptide, a CS1/SLAMF7 polypeptide, a B cell maturation antigen (BCMA) polypeptide, a CD38 polypeptide, and a CD123 polypeptide.

The presently disclosed subject matter also relates in some embodiments to T cells armed with a T-BiAb of the presently disclosed subject matter.

The presently disclosed subject matter also relates in some embodiments to methods for treating tumors and/or cancers. In some embodiments, the methods comprise, consist essentially of, or consist of contacting a tumor and/or a cancer with an effective amount of a composition comprising at least one T-BiAb of the presently disclosed subject matter, at least one T cell armed with a T-BiAb of the presently disclosed subject matter, or any combination thereof. In some embodiments, the tumor and/or the cancer is selected from the group consisting of a breast tumor and/or cancer, a pancreatic tumor and/or cancer, a prostate tumor and/or cancer, or a glioblastoma.

The presently disclosed subject matter also relates in some embodiments to methods for treating diabetes. In some embodiments, the methods comprise, consist essentially of, or consist of contacting a R-cell in a subject with an effective amount of a composition comprising one or more T-BiAbs, wherein each T-BiAb comprises a first binding moiety that binds to CD34 or CD45 and a second binding moiety that binds to a myosin light chain (MLC) polypeptide, wherein the first binding moiety is a single chain variable fragment (scFv) and the second binding moiety is a monoclonal antibody, and further wherein the variable light (V_{L}) and variable heavy (V_{H}) chains of the first binding moiety are directly linked as a single chain to the second binding moiety at the N-terminus or the C-terminus of the light chain or the heavy chain sequence of the second binding moiety. In some embodiments, the T-BiAb is bound to a stem cell.

The presently disclosed subject matter also relates in some embodiments to methods for arming and isolating stem cells. In some embodiments, the methods comprise, consist essentially of, or consist of contacting a stem cell with a T-BiAb comprising a first binding moiety that binds to CD34 or CD45 and a second binding moiety that binds to a myosin light chain (MLC) polypeptide, wherein the first binding moiety is a single chain variable fragment (scFv) and the second binding moiety is a monoclonal antibody, and further wherein the variable light (V_{L}) and variable heavy (V_{H}) chains of the first binding moiety are directly linked as a single chain to the second binding moiety at the N-terminus or the C-terminus of the light chain or the heavy chain sequence of the second binding moiety.

The presently disclosed subject matter also relates in some embodiments to methods for activating T cells. In some embodiments, the methods comprise, consist essentially of, or consist of contacting a T cell with a tetravalent bispecific antibody (T-BiAb) in an amount sufficient to activate the T cell, wherein the T-BiAb comprises, consists of, or consisting of a first binding moiety and a second binding moiety, and further wherein (i) the first binding moiety is a single chain variable fragment (scFv) comprising a variable light (V_{L}) chain and a variable heavy (V_{H}) chain; (ii) the second binding moiety is a monoclonal antibody comprising a light chain and a heavy chain; and (iii) the variable light (V_{L}) chain and the variable heavy (V_{H}) chain of the first binding moiety are directly linked as a single chain to the second binding moiety at the N-terminus or the C-terminus of the light chain or the heavy chain sequence of the second binding moiety to thereby generate an activated T cell. In some embodiments, the scFv binds to an CD3 polypeptide, optionally wherein the scFv is an scFv of an OKT3 monoclonal antibody. In some embodiments, the scFv binds to a CD3 polypeptide and the second binding moiety binds to a tumor-associated antigen (TAA). In some embodiments, the TAA is a polypeptide selected from the group consisting of an ERBB family member polypeptide, optionally an epidermal growth factor receptor (EGFR/ERBB1) polypeptide, a HER2/ERBB2 polypeptide, a HER3/ERBB3 polypeptide, a HER4/ERBB4 polypeptide, a disialoganglioside 2 (GD2) polypeptide, a MAG-1 polypeptide, a CD19 polypeptide, a CD20 polypeptide, a CD22 polypeptide, a CD30 polypeptide, a CD33 polypeptide, a CD34 polypeptide, a CS1/SLAMF7 polypeptide, a B cell maturation antigen (BCMA) polypeptide, a CD38 polypeptide, and a CD123 polypeptide. In some embodiments, the T cell is derived from a peripheral blood mononuclear cell (PBMC) or a tumor infiltrating T cell. In some embodiments, the T cell is a modified T cell that expresses a chimeric antigen receptor (CAR), optionally wherein the CAR is encoded by a transgene. In some embodiments, the activated T cell is characterized by a CD4⁺/CD25⁺/FoxP3⁺ T regulatory (Treg) phenotype.

In some embodiments, the presently disclosed method further comprises converting the activated T cells to a CD4⁺/CD25⁺/FoxP3⁺ T regulatory (Treg) cell or a CAR-T cell.

The presently disclosed subject matter also relates in some embodiments to compositions for use in treating tumors and/or cancers. In some embodiments, the compositions comprise, consist essentially of, or consisting of (a) at least one tetravalent bispecific antibody (T-BiAb) comprising, consisting essentially of, or consisting of a first binding moiety and a second binding moiety, wherein the first binding moiety is a single chain variable fragment (scFv) comprising a variable light (V_{L}) chain and a variable heavy (V_{H}) chain, the second binding moiety is a monoclonal antibody comprising a light chain and a heavy chain, and the variable light (V_{L}) chain and the variable heavy (V_{H}) chain of the first binding moiety are directly linked as a single chain to the second binding moiety at the N-terminus or the C-terminus of the light chain or the heavy chain sequence of the second binding moiety; (b) at least one T cell armed with the at least on T-BiAb; or (c) any combination thereof.

The presently disclosed subject matter also relates in some embodiments to compositions for use in treating diabetes. In some embodiments, the compositions comprise, consist essentially of, or consist of a tetravalent bispecific antibody (T-BiAb) comprising, consisting essentially of, or consisting of a first binding moiety and a second binding moiety, wherein the first binding moiety is a single chain variable fragment (scFv) comprising a variable light (V_{L}) chain and a variable heavy (V_{H}) chain, the second binding moiety is a monoclonal antibody comprising a light chain and a heavy chain, and the variable light (V_{L}) chain and the variable heavy (V_{H}) chain of the first binding moiety are directly linked as a single chain to the second binding moiety at the N-terminus or the C-terminus of the light chain or the heavy chain sequence of the second binding moiety.

The presently disclosed subject matter also relates in some embodiments to compositions for use in activating T cells. In some embodiments, the compositions comprise, consist essentially of, or consist of a tetravalent bispecific antibody (T-BiAb) comprising, consisting essentially of, or consisting of a first binding moiety and a second binding moiety, wherein the first binding moiety is a single chain variable fragment (scFv) comprising a variable light (V_{L}) chain and a variable heavy (V_{H}) chain, the second binding moiety is a monoclonal antibody comprising a light chain and a heavy chain, and the variable light (V_{L}) chain and the variable heavy (V_{H}) chain of the first binding moiety are directly linked as a single chain to the second binding moiety at the N-terminus or the C-terminus of the light chain or the heavy chain sequence of the second binding moiety.

In some embodiments of the compositions for use of the presently disclosed subject matter, the scFv binds to a CD3 polypeptide and the second binding moiety binds to a tumor-associated antigen (TAA). In some embodiments, the scFv that binds to the CD3 polypeptide is an scFv of an OKT3 monoclonal antibody. In some embodiments, the TAA is a polypeptide selected from the group consisting of an ERBB family member polypeptide, optionally an epidermal growth factor receptor (EGFR/ERBB1) polypeptide, a HER2/ERBB2 polypeptide, a HER3/ERBB3 polypeptide, a HER4/ERBB4 polypeptide, a disialoganglioside 2 (GD2) polypeptide, a MAG-1 polypeptide, a CD19 polypeptide, a CD20 polypeptide, a CD22 polypeptide, a CD30 polypeptide, a CD33 polypeptide, a CD34 polypeptide, a CS1/SLAMF7 polypeptide, a B cell maturation antigen (BCMA) polypeptide, a CD38 polypeptide, and a CD123 polypeptide. In some embodiments, the T cell is derived from a peripheral blood mononuclear cell (PBMC) or a tumor infiltrating T cell. In some embodiments, the T cell comprises a transgene that encodes a chimeric antigen receptor.

The presently disclosed subject matter also relates in some embodiments to compositions for use in arming and isolating stem cells. In some embodiments, the compositions comprise, consist essentially of, or consist of a T-BiAb comprising a first binding moiety that binds to CD34 or CD45 and a second binding moiety that binds to a myosin light chain (MLC) polypeptide, wherein the first binding moiety is a single chain variable fragment (scFv) comprising a variable light (V_{L}) chain and a variable heavy (V_{H}) chain and the second binding moiety is a monoclonal antibody, and further wherein the variable light (V_{L}) chain and variable heavy (V_{H}) chain of the first binding moiety are directly linked as a single chain to the second binding moiety at the N-terminus or the C-terminus of the light chain or the heavy chain sequence of the second binding moiety.

In some embodiments of the compositions for use of the presently disclosed subject matter, the T cell is characterized by a CD4⁺/CD25⁺/FoxP3⁺ T regulatory (Treg) phenotype.

In some embodiments of the compositions for use of the presently disclosed subject matter, the composition further comprises a pharmaceutically acceptable carrier, excipient, and/or diluent, optionally wherein the pharmaceutically acceptable carrier, excipient, and/or diluent is pharmaceutically acceptable for use in a human.

In some embodiments, the tetravalent bispecific antibody (T-BiAb) of the presently disclosed subject matter further comprises one or more pharmaceutically acceptable carriers, excipients, and/or diluents. In some embodiments, the pharmaceutically acceptable carrier, excipient, and/or diluent is pharmaceutically acceptable for use in a human.

In some embodiments, the armed T cell of presently disclosed subject matter further comprises a pharmaceutically acceptable carrier, excipient, and/or diluent, optionally wherein the pharmaceutically acceptable carrier, excipient, and/or diluent is pharmaceutically acceptable for use in a human.

In some embodiments, the presently disclosed subject matter also relates to uses of the tetravalent bispecific antibodies (T-BiAbs) of the presently disclosed subject matter and/or the armed T cells of the presently disclosed subject matter for the manufacture of a medicament for treating a tumor and/or a cancer in treating a tumor and/or a cancer, for treating diabetes, for activating a T cell, and/or for arming and/or isolating a stem cell.

Accordingly, it is an object of the presently disclosed subject matter to provide tetravalent bispecific antibodies (T-BiAbs). This and other objects are achieved in whole or in part by the presently disclosed subject matter.

Further, an object of the presently disclosed subject matter having been stated above, other objects and advantages of the presently disclosed subject matter will become apparent to those skilled in the art after a study of the following description, Figures, and EXAMPLES.

### BRIEF DESCRIPTION OF THE FIGURES

FIGS. 1A and 1B. SDS PAGE of T-BiAbs. FIG. 1A. rEGFRBi clone 1E2. Lanes contain 0.5 or 2 μL of purified BiAb adjacent to 2 g BSA. Molecular weight markers are indicated in kilodaltons. FIG. 1B. rHER2Bi (3× linker) and rHER2Bi (6× linker) T-BiAbs.

FIG. 2. Binding of rEGFRBi to normal donor ATC. Concentrations of rEGFRBi ranging from 50-800 ng/10⁶ ATC were incubated with ATC and stained for bound BiAb with PE-conjugated anti-human IgG by flow cytometry. MFI=median fluorescence intensity.

FIGS. 3A-3C. Cytotoxicity of rEGFR-, HER2-, and EGFR BATs from normal donors against SKBR3 (FIG. 3A), MDA MB231 (FIG. 3B), and MIA PaCa (FIG. 3C) cells at 10:1 E:T in overnight ⁵¹Cr-release assays. *p<0.05.

FIGS. 4A-4C. Cytotoxicity in U87 cells (FIG. 4A), SKBR3 cells (FIG. 4B), and MDA MB231 cells (FIG. 4C) of rEGFR BATs armed over a range of 1 to 400 ng rEGFRBi/10⁶ ATC at 10:1 E:T. HER2- and EGFR-BATs were armed at 50 ng BiAb/10⁶ ATC. *p<0.05.

FIGS. 5A-5D. Relative cytotoxicity of different BATs armed at 0.5 to 50 ng/10⁶ ATC. FIG. 5A. rEGFR BATs maintain high levels of cytotoxicity at 0.5 ng/million ATC against BxPC3 and MDAB231 cells. Values are the averages for 2 individual donors at 10:1 E:T. Black bars represent rEGFR BATs which maintain high cytotoxicity at all arming levels. FIG. 5B. rEGFRBi from 2 individual CHO-S clones (1E2 and 1B6) were used to arm 2 normal donor ATC at 10 ng and 1 ng BiAb/10⁶ ATC against MCF-7 and MIA PaCa cells at 10:1 E:T in overnight ⁵¹Cr-release assay. FIG. 5C. Cytotoxicity of rEGFR- and EGFR-BATs from normal donors against pancreatic cancer lines BxPC3 (2:1 E:T, n=4) using the xCELLigence Real-Time Cell Analysis (RTCA) assay. BATs were added to target cells and cytotoxicity assessed 18 hrs and 40 hrs later. *p<0.05. FIG. 5D. Cytotoxicity of rEGFR- and EGFR-BATs from normal donors against pancreatic cancer lines BxPC3 (2:1 E:T, n=4) and CFPAC (4:1 E:T, n=3) using the XCELLIGENCE® Real-Time Cell Analysis (RTCA) assay. BATs were added to target cells and cytotoxicity assessed 17-20 hours and 40 hours later. *p<0.05.

FIGS. 6A-6E. Relative cytokine release by EGFR-, HER2, rEGFR-BATs, and unarmed ATC against tumor cell lines armed with 50 ng BiAb/10⁶ ATC at E:T of 6:1 overnight. FIG. 6A. MIA PaCa-2. FIG. 6B. ASPC1. FIG. 6C. CFPAC-1. FIG. 6D. MDA MB231. FIG. 6E. PC3.

FIGS. 7A-7C. Relative expression of Th1 cytokines by rEGFR-BATs armed at 25 ng and 8 ng/10⁶ ATC and HER2 BATs armed at 50 ng/10⁶ ATC against prostate cancer cell line, PC3, at 12:1 (FIG. 7A), 6:1 (FIG. 7B), and 3:1 (FIG. 7C) E:T.

FIGS. 8A and 8B. FIG. 8A. Activation and expansion of normal donor T cells by OKT3 and rEGFRBi (clone 1E2). Normal donor PBMC were isolated by Ficoll-Hypaque density gradient centrifugation and activated by incubation with either OKT3 (Miltenyi) or rEGFRBi at 20 ng/10⁶ PMBC/mL in RPMI medium supplemented with 10% fetal bovine serum, 100 units penicillin/mL, 100 mcg streptomycin/mL, 2 mM glutamine and 100 IU IL-2 per 10⁶ cells. Cells were counted every 2 days and adjusted to 1.5×10⁶ cells/mL with fresh media with the addition of 100 IU IL-2 per 10⁶ cells. The number of ATC plotted represent the total number of cells after 14 days of culture. *p<0.05. FIG. 8B. Cytotoxicity of BATs prepared with ATC activated with OKT3 vs. rEGFRBi. ATC were armed with 50 ng CS1Bi (anti-CS1×anti-CD3)/10⁶ cells with and incubated with eFluor-450 labeled ARH77 cells at 1:1 E:T overnight. *p<0.05.

FIG. 9. SDS PAGE gel of anti-CD34×anti-MLC tetravalent BiAb (T-BiAb) proteins transiently expressed in 293S cells. Lanes 1 and 2. 380: 5B12×B12 (MLC); Lane 3. 385: 4C8×B12; Lane 4. 386: 2E10×B12; Lane 5. 387:A30×B12; Lane 6. a full length recombinant holobody as a control; Lane M=molecular weight markers; Lane 8. BSA (2 mg).

FIG. 10. Binding of anti-CD34×anti-MLC T-BiAbs to porcine cardiac myosin (PCM) by ELISA. T-BiAbs containing anti-CD34 variable regions from 5B12, 4C8 and 2E10 were incubated with serial 3-fold dilutions in wells coated with PCM. A monovalent BiAb (anti-CD34×anti-CD45) and total human IgG were also compared.

FIG. 11. CD34×MLC T-BiAbs to KG-1 cells. T-BiAbs 350, 385, and 386, human IgG and monovalent anti-CD34×CD45 BiAB 0173 were incubated with KG-1 cells at 100 ng/mL, stained with FITC anti-human Ig and analyzed with a Becton Dickenson FACSCAN flow cytometer.

FIG. 12. Binding of CD34×MLC T-BiAbs to CD34⁺ cord blood cells. T-BiAbs 350, 385, and 386, human IgG and monovalent anti-CD34×CD45 BiAB 0173 were incubated with CD34⁺ cord blood cells at 1 g/mL, stained with FITC anti-human Ig, and analyzed with a Becton Dickenson FACSCAN flow cytometer.

FIG. 13. Exemplary treatment schema for the injection of BiAb-armed and unarmed cord blood cells into STZ-treated diabetic NOD/SCID mice.

FIG. 14. Ejection fraction (EF) and glucose tolerance for combined groups of male and female diabetic mice treated with T-BiAb armed and unarmed (diamonds) CB cells. r-biab CD45 (squares)=recombinant monovalent anti-CD34×anti-CD45 BiAb-armed CD34 CB cells. r-biab CD34 (triangles)=tetravalent anti-CD34×anti-MLC (#380)-armed CD34⁺ CB cells.

FIG. 15. Ejection fraction (EF) and blood glucose levels of female mice treated with CB CD34⁺ cells. r-biab CD45 (squares)=recombinant monovalent anti-CD34×anti-CD45 BiAb-armed CD34 CB cells. r-BiAb CD34 (triangles)=tetravalent anti-CD34×anti-MLC (#380)-armed CD34⁺ CB cells. Diamonds correspond to unarmed controls.

### BRIEF DESCRIPTION OF THE SEQUENCE LISTING

SEQ ID NO: 1 is the nucleotide sequence of Sequence 1, an exemplary OKT3-EGFR heavy chain variable region (V_{H}) Xba1-Nhe1 cloning cassette. It includes nucleotides that encode, from 5′ to 3′, the OKT3 light chain variable region, a G4S₆ peptide linker, the OKT3 heavy chain variable region, a G4S₅ peptide linker, and the ERBITUX® heavy chain variable region.

SEQ ID NO: 2 is the amino acid sequence encoded by SEQ ID NO: 1.

SEQ ID NO: 3 is the nucleotide sequence of Sequence 2, an exemplary anti-EGFR kappa light chain (V_{κ}) Xba1-BsiW1-BamH1 cloning cassette derived from the sequence of ERBITUX®.

SEQ ID NO: 4 is the amino acid sequence encoded by SEQ ID NO: 3.SEQ ID NO: 5 is the amino acid sequence of an exemplary pentamer upon which linker peptides of the presently disclosed subject matter can be based. In SEQ ID NO: 5, the fourth amino acid can be glycine or threonine.

SEQ ID NOs: 6 and 7 are exemplary species of the pentamers of SEQ ID NO: 5, wherein the fifth amino acid is glycine in SEQ ID NON: 6 and threonine in SEQ ID NO: 7.

SEQ ID NO: 8 is the amino acid sequence of an exemplary linker peptide of the presently disclosed subject matter containing five copies of SEQ ID NO: 5 concatemerized. In SEQ ID NO: 8, each of the fourth, ninth, fourteenth, nineteenth, and twenty-fourth amino acids can independently be selected from the group consisting of glycine and threonine.

SEQ ID NOs: 9 and 10 are exemplary species of the exemplary linker peptide of SEQ ID NO: 8, wherein the fourth, ninth, fourteenth, nineteenth, and twenty-fourth amino acids are all glycines in SEQ ID NO: 9 and the fourth, ninth, nineteenth, and twenty-fourth amino acids are all glycines and the fourteenth amino acid is a threonine in SEQ ID NO: 10.

SEQ ID NO: 11 is the amino acid sequence of an exemplary linker peptide of the presently disclosed subject matter containing six copies of SEQ ID NO: 5 concatemerized. In SEQ ID NO: 11, each of the fourth, ninth, fourteenth, nineteenth, twenty-fourth, and twenty-ninth amino acids can independently be selected from the group consisting of glycine and threonine.

SEQ ID NOs: 12 and 13 are exemplary species of the exemplary linker peptide of SEQ ID NO: 11, wherein the fourth, ninth, fourteenth, nineteenth, twenty-fourth, and twenty-ninth amino acids are all glycines in SEQ ID NO: 12 and the fourth, ninth, nineteenth, twenty-fourth, and twenty-ninth amino acids are all glycines and the fourteenth amino acid is a threonine in SEQ ID NO: 10.

SEQ ID NOs: 14-22 are the predicted complementarity determining regions (CDRs) of the exemplary OKT3-EGFR embodiment of the presently disclosed subject matter. More particularly, SEQ ID NOs: 14-16 correspond to light chain CDRs 1-3 of OKT3, respectively; SEQ ID NOs: 17-19 correspond to heavy chain CDRs 1-3 of OKT3, respectively; and SEQ ID NOs: 20-22 correspond to heavy chain CDRs of ERBITUX®, respectively.

SEQ ID NOs: 23-25 are the predicted CDRs 1-3, respectively, of the exemplary ERBITUX® light chain of SEQ ID NO: 4.

### BRIEF DESCRIPTION OF THE TABLES

Table 1 summarizes amino acid codes and functionally equivalent codons.

Table 2 summarizes exemplary conservative amino acid substitutions.

Table 3 shows median fluorescence intensity (1,000) of ERBITUX® and rEGFRBi bound to tumor cells.

Table 4 shows the ratios of increased secretion of the cytokines GM-CSF, IFN-γ, TNF-α, and GrB by rEGFR- vs. EGFR BATs (pancreatic cancer), HER2-BATs (breast and prostate cancer), and GD2 BATs (neuroblastoma) P=p value, Student's t-test.

Table 5 shows expansion of T cells from PBMC activated by OKT3 or rEGFRBi at different doses and on days 0, 4, 6, 8, 10, 12, and 14.

Table 6 summarizes scFv and N-terminus linker sequences for various constructs of the presently disclosed subject matter.

Table 7 shows targets of clinical stage T cell redirected therapeutics from Strohl & Naso, 2019.

Table 8 summarizes the components of certain exemplary MLCBiAB constructs of the presently disclosed subject matter.

### DETAILED DESCRIPTION

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in Table 1:

TABLE 1
Amino Acid Codes and Functionally Equivalent Codons

| | 3-Letter | 1-Letter | |
|---|---|---|---|
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide. Amino acids have the following general structure:

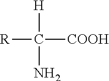

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in Table 2:

TABLE 2
Exemplary Conservative Amino Acid Substitutions

| Group | Characteristics | Amino Acids |
|---|---|---|
| A. | Small aliphatic, nonpolar, or slightly | Ala, Ser, Thr, Pro, Gly |
| | polar residues | |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990a, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Altschul et al., 1997) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane, 1988 for a description of immunoassay formats and conditions that can be used to determine specific or selective immunoreactivity. See also the EXAMPLES set forth herein below for additional formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; in some embodiments in 7% (SDS), 0.5 M NaPO₄, 1 mM EDTA at 50° C. with washing in 1×SSC, 0.1% SDS at 50° C.; in some embodiments 7% SDS, 0.5 M NaPO₄, 1 mM EDTA at 50° C. with washing in 0.5×SSC, 0.1% SDS at 50° C.; and more in some embodiments in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Exemplary Embodiments

In some embodiments, the presently disclosed subject matter relates to tetravalent bispecific antibodies (T-BiAbs) comprising a first binding moiety and a second binding moiety, wherein the first binding moiety is a single chain variable fragment (scFv) and the second binding moiety is a monoclonal antibody. In some embodiments, the variable light (V_{L}) chain and the variable heavy (V_{H}) chain of the first binding moiety are directly linked as a single chain to the second binding moiety at the N-terminus or the C-terminus of the light chain or the heavy chain sequence of the second binding moiety. Thus, in some embodiments the presently disclosed subject matter is based on the structure of the GGGGS (G4S; SEQ ID NO: 6) repeat unit linker joining the V_{H} and V_{L} chains of the first antibody. In some embodiments, an scFv containing a pentamer repeat [(G4S)₅] or a hexamer repeat [(G4S)₆] is made with the configuration V_{L}-(G4S)_{5/6}-V_{H} In some embodiments, the configuration is reversed: V_{H}-(G4S)_{5/6}-V_{L}. In some embodiments, the pentamer repeat has the amino acid sequence GGGXSGGGXSGGGXSGGGXSGGGXS (SEQ ID NO: 8), wherein each X is independently glycine (G) or threonine (T), which in some embodiments is GGGGSGGGGSGGGGSGGGGSGGGGS (SEQ ID NO: 9) and in some embodiments is GGGGSGGGGSGGGTSGGGGSGGGGS (SEQ ID NO: 10). In some embodiments, the hexamer repeat has the amino acid sequence GGGXSGGGXSGGGXSGGGXSGGGXSGGGXS (SEQ ID NO: 11), wherein each X is independently glycine (G) or threonine (T), which in some embodiments is GGGGSGGGGSGGGGSGGGGSGGGGSGGGGS (SEQ ID NO: 12) and in some embodiments is GGGGSGGGGSGGGTSGGGGSGGGGSGGGGS (SEQ ID NO: 13).

The scFv can be fused to the N- or C-terminus of the heavy chain of the second antibody or to the N- or C-terminus of the light chain of the second antibody. Heavy chain isotypes can be selected according to desired characteristics for secondary functions, stability, or species specificity (Vidarsson et al., 2014; Bruhns & Jonsson, 2015; Saunders, 2019). Gene expression vectors for the scFv-fusion construct are co-transfected with an expression vector for the complementary chain of the second antibody (to provide paired V_{H} and V_{L} chains) into an appropriate cell line for expression and purification. The number of (G4S) units in the linker for the scFv can range from 3-6.

The scFv of the first antibody is linked to the N- or C-terminus of a chain of the second antibody using a second linker (G4S)₅ (i.e., SEQ ID NO: 9). In some cases a modification is made to provide for a G to T substitution in one of the (G4S) repeats (SEQ ID NO: 8), which in some embodiments is GGGGSGGGGSGGGTSGGGGSGGGGS (SEQ ID NO: 10).

In some cases the expression vectors are transfected into, e.g., 293S cells, for transient expression. In other cases, the expression vectors incorporate genes for selectable markers and are transfected into, e.g., CHO-S cells for isolation of stably expressing clones. Cell-free supernatants are used to isolate purified antibodies via, e.g., Protein A, Protein G, or Protein L column chromatography. The purified proteins are analyzed for size and structure using non-denaturing and denaturing sodium dodecyl sulfate (SDS) polyacrylamide gel electrophoresis (PAGE). Protein is quantitated using, e.g., Bradford colorimetric assay using a known concentration of monoclonal antibody as a standard. Binding studies to assess affinities of the T-BiAb specificities are performed, e.g., through binding to surface antigens of cells using flow cytometry, or to target proteins using enzyme-linked immunoassay (ELISA). Depending on the nature of the binding specificities, functional assays can be performed, e.g., cell-mediated cytotoxicity, cytokine expression, tissue targeting, tissue repair, and cell homing, among others.

Thus, in some embodiments the presently disclosed subject matter relates to tetravalent bispecific antibodies (T-BiAbs) comprising a first binding moiety and a second binding moiety, wherein the first binding moiety is a single chain variable fragment (scFv) and the second binding moiety is a monoclonal antibody, and further wherein the variable light (V_{L}) and variable heavy (V_{H}) chains of the first binding moiety are directly linked as a single chain to the second binding moiety at the N-terminus or the C-terminus of the light chain or the heavy chain sequence of the second binding moiety. As used herein, the phrase “binding moiety” refers to the antigen-binding portion of an antibody (i.e., a paratope).

In some embodiments of the presently disclosed subject matter, the variable light (V_{L}) and variable heavy (V_{H}) chains of the first binding moiety are linked to each other as a single polypeptide chain via a peptide linker. In some embodiments, the peptide linker comprises, consists essentially of, or consists of the amino acid sequence GGGGS (SEQ ID NO: 6), optionally wherein the peptide linker comprises, consists essentially of, or consists of a concatemer of 3-6 copies of the amino acid sequence GGGGS (SEQ ID NO: 6). In some embodiments, at least one of the copies of the amino acid sequence GGGGS (SEQ ID NO: 6) includes an amino acid substitution to GGGTS (SEQ ID NO: 7), optionally wherein the peptide linker comprises, consists essentially of, or consists of the amino acid sequence GGGGSGGGGSGGGTSGGGGSGGGGS (SEQ ID NO: 10) or GGGGSGGGGSGGGTSGGGGSGGGGSGGGGS (SEQ ID NO: 13).

In some embodiments, the variable light (V_{L}) and variable heavy (V_{H}) chains of the first binding moiety (e.g., the scFv) are linked to each other in a configuration selected from the group consisting of V_{L}-(G4S)ₓ-V_{H} and V_{H}-(G4S)ₓ-V_{L}, wherein G4S is the amino acid sequence GGGGS (SEQ ID NO: 6) or a threonine-containing variant thereof (e.g., GGGTS; SEQ ID NO: 7) and x is 3-6. In some embodiments, one or more of the GGGGS (SEQ ID NO: 6) monomers in the concatemer (G4S), includes a glycine to threonine substitution, optionally wherein the threonine is substituted at the position of the C-terminal glycine of the monomer (e.g., GGGTS; SEQ ID NO: 7).

In some embodiments, the first binding moiety (e.g., the scFv) binds to a CD3 polypeptide. In some embodiments, the first binding moiety (e.g., the scFv) that binds to the CD3 polypeptide is an OKT3 monoclonal antibody or an scFv fragment derived therefrom. It is noted, however, that the first binding moiety (e.g., the scFv) that binds to the CD3 polypeptide need not be based on the OKT3 monoclonal antibody. Other commercially available anti-CD3 antibodies include those from BioLegend, Inc. (San Diego, California, United States of America), ThermoFisher Scientific (Waltham, Massachusetts, United States of America), Abcam plc (Waltham, Massachusetts, United States of America), etc., any of which can be employed in the compositions and methods of the presently disclosed subject matter. See also U.S. Pat. No. 8,551,478, the content of which is incorporated by reference in its entirety.

In some embodiments, the second binding moiety binds to a tumor-associated antigen (TAA). Various tumor-associated antigens (TAAs) are known, and any TAA can be employed in the compositions and methods of the presently disclosed subject matter. Exemplary TAAs include, but are not limited to 5-alpha reductase, alpha-fetoprotein, AM-1, APC, April, BAGE, beta-catenin, Bcl12, bcr-abl, CA-125, CASP-8/FLICE, Cathepsins, CD19, CD20, CD21, CD23, CD22, CD33 CD35, CD44, CD45, CD46, CD5, CD52, CD55, CD59, CDC127, CDK4, CEA, c-myc, Cox-2, DCC, DcR3, E6/E7, CGFR, EMBP, Dna78, famesyl transferase, FGF8b, FGF8a, FLK-1/KDR, folic acid receptor, G250, GAGE-family, gastrin 17, gastrin-releasing hormone, GD2/GD3/GM2, GnRH, GnTV, GP1, gp100/Pmel17, gp-100-in4, gp15, gp75/TRP-1, hCG, heparanse, Her2/neu, HMTV, Hsp70, hTERT, IGFR1, IL-13R, iNOS, Ki67, KIAA0205, K-ras, H-ras, N-ras, KSA, LKLR-FUT, MAGE-family, mammaglobin, MAP17, melan-A/MART-1, mesothelin, MIC A/B, MT-MMPs, mucin, NY-ESO-1, osteonectin, p15, P170/MDR1, p53, p97/melanotransferrin, PAI-1, PDGF, uPA, PRAME, probasin, progenipoientin, PSA, PSM, RAGE-1, Rb, RCAS1, SART-1, SSX-family, STAT3, STn, TAG-72, TGF-alpha, TGF-beta, Thymosin-beta-15, TNF-alpha, TRP-1, TRP-2, tyrosinase, VEGF, ZAG, p16INK4, and glutathione-S-transferase. In some embodiments, a TAA is a polypeptide selected from the group consisting of an ERBB family member polypeptide, optionally an epidermal growth factor receptor (EGFR/ERBB1) polypeptide, a HER2/ERBB2 polypeptide, a HER3/ERBB3 polypeptide, a HER4/ERBB4 polypeptide, a disialoganglioside 2 (GD2) polypeptide, MAG-1, CD19, CD20, CD22, CD30, CD33, CD34, CS1/SLAMF7, B cell maturation antigen (BCMA), CD38, and CD123. In some embodiments, the tumor-associated antigen is a human tumor-associated antigen.

Also provided are T cells and/or other immune effector cells (e.g., NK cells, monocytes, polymorphonuclear cells, B cells, dendritic cells, etc.) armed with one or more T-BiAbs as described herein.

Also provided are compositions for use in various methods of the presently disclosed subject matter. These methods include, but are not limited to methods for treating tumors and/or cancers, for treating diabetes, for activating T cells, and for arming and isolating stem cells. In various embodiments, the compositions comprise, consist essentially of, or consist of:
(a) at least one tetravalent bispecific antibody (T-BiAb) comprising, consisting essentially of, or consisting of a first binding moiety and a second binding moiety, wherein the first binding moiety is a single change variable fragment (scFv) comprising a variable light (V_{L}) chain and a variable heavy (V_{H}) chain, the second binding moiety is a monoclonal antibody comprising a light chain and a heavy chain, and the variable light (V_{L}) chain and the variable heavy (V_{H}) chain of the first binding moiety are directly linked as a single chain to the second binding moiety at the N-terminus or the C-terminus of the light chain or the heavy chain sequence of the second binding moiety;
(b) at least one T cell armed with the at least on T-BiAb; or
(c) any combination thereof.

In some embodiments, the compositions for use of the presently disclosed subject matter comprise an scFv that binds to a CD3 polypeptide, a second binding moiety that binds to a tumor-associated antigen (TAA), or both. In some embodiments, the scFv that binds to the CD3 polypeptide is an scFv of an OKT3 monoclonal antibody. In some embodiments, the TAA is a polypeptide selected from the group consisting of an ERBB family member polypeptide, optionally an epidermal growth factor receptor (EGFR/ERBB1) polypeptide, a HER2/ERBB2 polypeptide, a HER3/ERBB3 polypeptide, a HER4/ERBB4 polypeptide, a disialoganglioside 2 (GD2) polypeptide, a MAG-1 polypeptide, a CD19 polypeptide, a CD20 polypeptide, a CD22 polypeptide, a CD30 polypeptide, a CD33 polypeptide, a CD34 polypeptide, a CS1/SLAMF7 polypeptide, a B cell maturation antigen (BCMA) polypeptide, a CD38 polypeptide, and a CD123 polypeptide. In some embodiments, the TAA is a human polypeptide.

Furthermore, the T-BiAbs and/or the T cells and/or other immune effector cells described herein can be employed in various treatment methods as also described herein. In some embodiments, methods for treating a tumor and/or a cancer are provided. In some embodiments, the methods comprise, consist essentially of, or consist of contacting a tumor and/or a cancer with a composition comprising an effective amount of at least one T-BiAb as described herein, a T cell and/or other immune effector cell armed with at least one T-BiAb as described herein, or any combination thereof. As used herein, the phrase “a composition comprising an effective amount of at least one T-BiAb” contemplates administering compositions in single doses or in multiple doses, wherein the effective amount refers to the amount of the single dose or of the cumulative doses that results in a desired treatment outcome.

Depending on the TAA that is targeted by the T-BiAb, various tumors and/or cancers can be targeted by the compositions and methods of the presently disclosed subject matter. By way of example only, in some embodiments the tumor and/or the cancer is selected from the group consisting of a breast tumor and/or cancer, a pancreatic tumor and/or cancer, a prostate tumor and/or cancer, or a glioblastoma.

In some embodiments, the T-BiAbs target not TAAs but other antigens of biological significance. Thus, in some embodiments the presently disclosed subject matter provides methods for treating diabetes, autoimmune diseases, inflammatory conditions, and other diseases, disorders, and conditions that can be treated with antibodies generally.

Accordingly, in some embodiments the presently disclosed methods comprise, consist essentially of, or consist of contacting a R-cell in a subject with a composition comprising an effective amount of at least one T-BiAb, wherein the at least one T-BiAb comprises a first binding moiety that binds to CD34 or CD45 and a second binding moiety that binds to a myosin light chain (MLC) polypeptide, wherein the first binding moiety is a single change variable fragment (scFv) and the second binding moiety is a monoclonal antibody, and further wherein the variable light (V_{L}) and variable heavy (V_{H}) chains of the first binding moiety are directly linked as a single chain to the second binding moiety at the N-terminus or the C-terminus of the light chain or the heavy chain sequence of the second binding moiety. In some embodiments, at least one of the T-BiAbs is bound to a stem cell.

In some embodiments, the first binding moiety and/or the second binding moiety binds to an antigen that is present on a stem cell. As such, the presently disclosed T-BiAbs can be employed for method of arming and/or isolating stem cells. In some embodiments, the methods comprise, consist essentially of, or consist of contacting a stem cell with a T-BiAb comprising a first binding moiety that binds to CD34 or CD45 and a second binding moiety that binds to a myosin light chain (MLC) polypeptide, wherein the first binding moiety is a single chain variable fragment (scFv) and the second binding moiety is a monoclonal antibody, and further wherein the variable light (V_{L}) and variable heavy (V_{H}) chains of the first binding moiety are directly linked as a single chain to the second binding moiety at the N-terminus or the C-terminus of the light chain or the heavy chain sequence of the second binding moiety. The binding of the first binding moiety and the second binding moiety thus tags the stem cell, and reagents that can be employed for affinity purifying the T-BiAbs/stem cell complex are known.

### Antibody Formats and Preparation Thereof

Antibodies directed against proteins, polypeptides, or peptide fragments thereof of the presently disclosed subject matter may be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab.

For the preparation of monoclonal antibodies, any technique which provides for the production of antibody molecules by continuous cell lines in culture may be utilized. For example, the hybridoma technique originally developed in 1975 by Kohler and Milstein (Kohler & Milstein, 1975), the trioma technique, the human B-cell hybridoma technique (Kozbor & Roder, 1983), and the EBV-hybridoma technique (Cole et al., 1985) may be employed to produce human monoclonal antibodies. In some embodiments, monoclonal antibodies are produced in germ-free animals.

In accordance with the presently disclosed subject matter, human antibodies may be used and obtained by utilizing human hybridomas (Cote et al., 1983) or by transforming human B cells with EBV virus in vitro (Cole et al., 1985). Furthermore, techniques developed for the production of “chimeric antibodies” (Morrison et al., 1984; Neuberger et al., 1984; Takeda et al., 1985) by splicing the genes from a mouse antibody molecule specific for epitopes of SLLP polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

Various techniques have been developed for the production of antibody fragments of humanized antibodies. Traditionally, these fragments were derived via proteolytic digestion of full-length antibodies (see e.g., Morimoto & Inouye, 1992; Brennan et al., 1985). However, these fragments can now be produced directly by recombinant host cells. Alternatively, Fab′-SH fragments can be directly recovered from E. coli and chemically coupled to form F(ab′)₂ fragments (Carter et al., 1992a). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See PCT International Patent Application Publication No. WO 1993/16185; U.S. Pat. Nos. 5,571,894; 5,587,458. The antibody fragment may also be a “linear antibody”, e.g., as described in U.S. Pat. No. 5,641,870, for example. Such linear antibody fragments may be monospecific or bispecific.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see e.g., U.S. Pat. Nos. 4,975,369; 5,075,431; 5,081,235; 5,169,939; 5,202,238; 5,204,244; 5,231,026; 5,292,867; 5,354,847; 5,472,693; 5,482,856; 5,491,088; 5,500,362; and 5,502,167). Detailed methods for preparation of chimeric (humanized) antibodies can be found in U.S. Pat. No. 5,482,856. A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see e.g., Jones et al., 1986; Riechmann et al., 1988; Presta, 1992, PCT International Patent Application Publication No. WO 92/02190, U.S. Patent Application Publication No. 2006/0073137, and U.S. Pat. Nos. 5,225,539; 5,530,101; 5,585,089; 5,693,761; 5,693,762; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; and 6,750,325.

In some embodiments, the presently disclosed subject matter provides for fully human antibodies. Human antibodies consist entirely of characteristically human polypeptide sequences. The human antibodies of this presently disclosed subject matter can be produced in using a wide variety of methods (see e.g., U.S. Pat. No. 5,001,065, for review).

Typically, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., 1986; Riechmann et al., 1988; Verhoeyen et al., 1988), by substituting hypervariable region sequences for the corresponding sequences of a human “acceptor” antibody. Accordingly, such “humanized” antibodies are chimeric antibodies (see e.g., U.S. Pat. Nos. 4,816,567 and 5,482,856) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

Another method for making humanized antibodies is described in U.S. Patent Application Publication No. 2003/0017534, wherein humanized antibodies and antibody preparations are produced from transgenic non-human animals. The non-human animals are genetically engineered to contain one or more humanized immunoglobulin loci that are capable of undergoing gene rearrangement and gene conversion in the transgenic non-human animals to produce diversified humanized immunoglobulins.

In some embodiments, the choice of human variable domains, both light and heavy, to be used in making the humanized antibodies is very important to reduce antigenicity. According to the so-called “best-fit” method, the sequence of the variable domain of a rodent antibody is screened against a library of known human variable-domain sequences or a library of human germline sequences. The human sequence that is closest to that of the rodent can then be accepted as the human framework region for the humanized antibody (Sims et al., 1993; Chothia & Lesk, 1987). Another method uses a particular framework region derived from the consensus sequence of all human antibodies of a particular subgroup of light or heavy chains. The same framework may be used for several different humanized antibodies (Carter et al., 1992b; Presta et al., 1993). Other methods designed to reduce the immunogenicity of the antibody molecule in a human patient include veneered antibodies (see e.g., U.S. Pat. No. 6,797,492 and U.S. Patent Application Publication Nos. 2002/0034765 and 2004/0253645) and antibodies that have been modified by T cell epitope analysis and removal (see e.g., U.S. Patent Application Publication No. 2003/0153043 and U.S. Pat. No. 5,712,120).

It is important that when antibodies are humanized they retain high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The antibody moieties of this presently disclosed subject matter can be single chain antibodies.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Patent Application Publication No. 2003/0022244.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments.

In some embodiments, the specific binding molecule is a single-chain variable analogue (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies, tri-specific such as triabodies and tetra-specific such as tetrabodies when each scFv in the dimer, trimer, or tetramer has a different specificity. Diabodies, triabodies and tetrabodies can also be monospecific, when each scFv in the dimer, trimer, or tetramer has the same specificity.

In some embodiments, techniques described for the production of single-chain antibodies (U.S. Pat. No. 4,946,778, incorporated by reference herein in its entirety) are adapted to produce protein-specific single-chain antibodies. In some embodiments, the techniques described for the construction of Fab expression libraries (Huse et al., 1989) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which bind the antigen therefrom at any epitopes present therein.

Monoclonal antibodies directed against full length or peptide fragments of a protein or peptide may be prepared using any well known monoclonal antibody preparation procedures, such as those described, for example, in Harlow & Lane, 1988; Tuszynski et al., 1988). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Exemplary complementarity-determining region (CDR) residues or sequences and/or sites for amino acid substitutions in framework region (FR) of such humanized antibodies having improved properties such as, e.g., lower immunogenicity, improved antigen-binding or other functional properties, and/or improved physicochemical properties such as, e.g., better stability, are provided.

The presently disclosed subject matter encompasses more than the specific fragments and humanized fragments disclosed herein. In some embodiments, the antibody is selected from the group consisting of a single chain antibody, a monoclonal antibody, a bi-specific antibody, a chimeric antibody, a synthetic antibody, a polyclonal antibody, or a humanized antibody, or active fragments or homologs thereof.

A nucleic acid encoding the monoclonal antibody obtained using the procedures described herein may be cloned and sequenced using technology that is available in the art, and is described, for example, in Wright et al., 1992) and the references cited therein. Further, the antibody of the presently disclosed subject matter may be “humanized” using the technology described in Wright et al., 1992 and in the references cited therein, and in Gu et al., 1997.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Green & Sambrook, 2012.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton & Barbas, 1994). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions may be synthesized such that they include nearly all possible specificities (Barbas, 1995; de Kruif et al., 1995).

In the production of antibodies, screening for the desired antibody can be accomplished by techniques known in the art, e.g., ELISA (enzyme-linked immunosorbent assay). Antibodies generated in accordance with the presently disclosed subject matter may include, but are not limited to, polyclonal, monoclonal, chimeric (i.e., “humanized”), and single chain (recombinant) antibodies, Fab fragments, and fragments produced by a Fab expression library.

It is common in the field of recombinant humanized antibodies to graft murine CDR sequences onto a well-established human immunoglobulin framework previously used in human therapies such as the framework regions of Herceptin [Trastuzumab].

In some embodiments, when used in vivo for therapy, the antibodies of the subject presently disclosed subject matter are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

In some embodiments, for parenteral administration, the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

### Pharmaceutical Compositions and Administration

The presently disclosed subject matter is also directed to methods of administering the compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method for treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the peptides of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The peptides of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments or drops), or as a buccal or nasal spray or aerosol. Parenteral administration is preferred. Particularly preferred parenteral administration methods include intravascular administration (e.g., intravenous bolus injection, intravenous infusion, intra-arterial bolus injection, intra-arterial infusion and catheter instillation into the vasculature), peri- and intra-target tissue injection (e.g., peri-tumoral and intra-tumoral injection), subcutaneous injection or deposition including subcutaneous infusion (such as by osmotic pumps), intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference.

Typically, dosages of the compound of the presently disclosed subject matter which may be administered to an animal, in some embodiments a human, range in amount from 1 g to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the EXAMPLES

Recombinant anti-EGFR light chain and OKT3 scFv-anti-EGFR heavy chain gene fusion constructs. The variable light and heavy gene sequences from the DrugBank amino acid entry for ERBITUX® were backtranslated using the most common homologous mouse sequences in the IMGT database. The variable light and heavy chain genes for humanized OKT3 were designed based on amino acid sequences in U.S. Pat. No. 5,885,573 (incorporated herein by reference in its entirety). The OKT3 scFv-Erbitux-heavy chain fusion gene was assembled in the order: variable light chain—(G4S)₆ linker-variable heavy chain—(G4S)₅ (with a change in repeat 4 to introduce a G to T substitution) linker—Erbitux heavy chain variable sequence—human IgG1 Fc. Coding regions for the Erbitux light and OKT3scFv-Erbitux fusion genes were then subcloned into the proprietary expression vector SwiMR (see U.S. Pat. No. 9,910,038, incorporated herein by reference in its entirety) which contains a selectable marker for puromycin or neomycin for the respective heavy and light chains. DNA and protein sequences of the antibody coding regions are shown in Sequences 1 and 2 (below).

Selection of stably expressing rEGFRBi clones. The plasmid vectors were linearized and used to transfect CHO suspension cells (CHO-S). The clones were selected in the presence of puromycin and neomycin and screened for antibody expression by ELISA. The highest expressing clones were expanded into shake flask cultures and cryopreserved.

BiAb Purification. rEGFRBi was purified from cell-free supernatants via Protein A chromatography. The BiAb samples were stored in 1× phosphate buffered saline (PBS) at −80° C., −20° C., or 4° C.

Cell lines. The following cell lines were obtained from ATCC and maintained in culture according to the vendor: breast cancer lines SKBR3, MCF-7, MDA-MB-231; pancreatic cancer lines MIA PaCa-2, CFPAC-1, AsPC-1, and BxPC-3; prostate cancer line PC3; and glioblastoma line U87.

Preparation of heteroconjugated BiAbs. Herceptin, Erbitux and OKT3 (Miltenyi or BioXcell) were obtained commercially. Antibodies were heteroconjugated with OKT3 as previously described in Sen et al., 2001 and Reusch et al., 2006. Briefly, OKT3 was crosslinked with Traut's reagent (Thermo Scientific), and anti-tumor antibodies crosslinked with sulfo-SMCC (Thermo Scientific) per manufacturer's instructions. After buffer exchange, equal amounts of each antibody pair (OKT3 and anti-tumor antibody) were mixed and rocked overnight at 4° C. to achieve heteroconjugation. The antibodies were characterized by SDS PAGE, quantitated using Bio-Rad Protein Assay Kit.

Flow cytometry. Approximately 0.5×10⁶ tumor cells or ATC were incubated with rEGFRBi, Erbitux or human IgG1 antibodies in 100 μL flow wash buffer (PBS, 0.2% BSA) for 30 minutes at 4° C. The cells were washed and incubated with Phycoerythrin (PE)-conjugated anti-human IgG (BioLegend 409303). Cells were analyzed by flow cytometry using an ACEA Biosciences NovoCyte flow cytometer and analyzed with NOVOEXPRESS® software.

Activated T lymphocytes. Peripheral blood mononuclear cells (PBMC) from normal subjects were obtained under University of Virginia (UVA) IRB #18904 under informed and written consent. PBMC were isolated by Ficoll-Hypaque (Lymphocyte Separation Medium from Corning) and stimulated with OKT3 at 20 ng/ml and expanded in RPMI-1640 containing 10% fetal calf serum and IL-2 (100 IU/ml) as described in reference 17 below. ATC were armed with the designated concentration of BiAbs for 15′ under rocking conditions at room temperature, washed with RPMI medium (with 10% fetal bovine serum) and resuspended at the required concentration for a given effector to target ratio (E:T).

Cytotoxicity assay: ⁵¹Cr-release assay. ⁵¹Cr-release assays were performed as previously described in reference 19 below. Briefly, between 10-20,000 target cells were seeded per well of 96-well plates in triplicate in 100 μL media and adhered overnight (or 48 hours) at 37° C. The cells were radioactively labeled with 2 μCi/well of ⁵¹Cr for 4-6 hours at 37° C. BATs were added at designated E:T ratios in 100 μL media. After 16-20 hours, 100 μL of cell-free supernatant was added to 100 μL liquid scintillation cocktail and counted in a Perkin Elmer MicroBeta scintillation counter. Wells without added effector cells were used as spontaneous release controls. Wells treated with 100 μL of 2% SDS were used as maximum release controls. Percent cytotoxicity was calculated as: [(experimental cpm−spontaneous release cpm)/(maximum release cpm)]×100.

Cytotoxicity assays using the xCELLigence Real Time Cell Analysis Multiple Plate (RTCA MP) system. Cytotoxicity assays of BATs against the pancreatic cancer cell lines BxPC3 and CFPAC were performed using the cell-impedance based xCELLigence RTCA MP system (ACEA Biosciences, Agilent) and the respective 96-well E-Plates (ACEA Biosciences, Agilent). A media-only impedance check was performed first by adding 50 μL of cell culture media to every well of the 96-well E-Plates and then measuring the background impedance as a unit of Cell Index. The cell lines were trypsinized, washed, and seeded at 8×10′ cells/well. Seeded plates were left at ambient temperature for 30 minutes to allow for even distribution and settling of tumor cells. Afterwards, the plates were transferred to the xCELLigence RTCA MP system in a humidified 37° C. O₂ incubator and incubated overnight. The RTCA MP system was programmed to collect Cell Index data points at 15 minute intervals. The next day, cryopreserved OKT3-activated T cells were thawed, washed twice, and armed with either a chemically conjugated OKT3×EGFR BiAb or rEGFRBi at 50 ng/1×10⁶ cells for 15 minutes to generate EGFR- and rEGFR BATs. Three wells of each target cell type were trypsinized and pooled for counting by hemocytometer or by flow cytometry (ACEA Biosciences Novocyte, Agilent). The BATs were washed with culture media and the cell density adjusted for the desired E:T ratio. The BATs and unarmed ATC were seeded onto the E-Plate at an E:T ratio of 2:1 or 4:1 and returned to the RTCA MP. Data acquisition was resumed and the E-Plate was incubated for continuous monitoring over 40 hours. Data output was normalized to a common Delta Time, which converted Cell Index to Delta Cell Index (DCI) and established a common starting point for each condition. The % cytotoxicity was calculated for 18 and 40 hour time points using the following equation:

$$\% Cytotoxicity = ( \frac{{DCI}_{Tumor Alone} - {DCI}_{Treated}}{{DCI}_{Tumor Alone}} ) \times 100$$

wherein DCI_{Tumor Alone} is the average Delta Cell Index between replicates of untreated tumor cells DCI_{Treated} is the average Delta Cell index between replicates of BATs- or ATC-treated wells.

Cytokine assays. Cell-free supernatants from cytotoxicity assays performed without ⁵¹Cr were collected and frozen at −20° C. until analyzed. Samples were diluted and analyzed using R&D Luminex kits.

### Introduction to the EXAMPLES

Chemotherapy (chemoT) resistance and immunosuppression provide major challenges to cancer therapy. Despite recent advances in chemotherapy (chemoT) and immunotherapy (IT), cancer still remains one of the leading causes of mortality. An estimated 1,762,450 new cases of cancer and 606,880 cancer deaths are projected in the U.S. in 2019 (see Siegel et al., 2019). Evolution of cancer cells and the tumor microenvironment (TME) inevitably lead to acquisition of therapy resistance to chemoT and/or IT. Adoptive cell therapies (ACTs) offer a promising new avenue in the anti-cancer treatment landscape, especially due to their targeting specificity and ability to mediate localized cytotoxicity and immunomodulation. The TME presents major challenges, in particular the variable expression of tumor associated antigens (TAA), clonal escape mechanisms, structural and physical barriers in tumor stromal cells that limit penetration of effector cells and chemotherapeutics, tumor-promoting factors produced by tumors, and the influence of myeloid derived suppressor cells (MDSC) and T regulatory cells (T_{REGs}). Expression of transforming growth factor β (TGFβ), interleukin-4 (IL-4), arginase, reactive oxygen species (ROS), and inhibitory ligands such as PD-L1, PD-L2, and CTLA4, act to suppress anti-tumor T cell responses. Despite the success of chimeric antigen receptor (CAR)-T cells against hematologic malignancies, the inhibitory mechanisms of the solid TME are more formidable than expected even though CAR-T cells have been engineered for increased potency and replicative capacity (Schmidts & Maus, 2018; Heyman & Yang, 2019). Although CAR-T cells are cytotoxic, proliferate, and secrete Th₁ cytokines, CAR-T become exhausted, and can cause life-threatening cytokine release syndrome (CRS), while the infusion of BiAbs alone is also associated with CRS. Only one BiAb, directed at CD19, is FDA approved. These results underscore the need to develop non-toxic effective targeting agents for solid tumors.

EGFR (ERBB-1 Receptor, HER1) as a target. EGFR is a receptor tyrosine kinase belonging to the EGF family of growth factors, and plays a central role in generation of several carcinomas. Overexpression of EGFR is seen in head and neck cancer, genito-urinary cancers, gastrointestinal malignancies, prostate carcinoma, breast carcinoma, thyroid carcinomas, salivary gland carcinomas, melanoma gastro-esophageal cancer, non-small cell lung cancer (NSCLC), and endometrial cancers (Salomon et al., 1995; Clauditz et al., 2013; Chiosea et al., 2015; Yan et al., 2015). EGFR overexpression is seen in advanced disease stage and is associated with poor prognosis (Yasui et al., 1988; Chung & Antoniades, 1992; Tan & Yu, 2007). Interventions targeting EGFR signaling pathways in solid tumors have shown improved patient outcomes. Several therapies including monoclonal antibodies and small molecule tyrosine kinase inhibitors are now FDA approved. In clinical trials, small molecule inhibitors that target the intracellular tyrosine kinase signaling pathways of EGFR, such as gefitinib (IRESSA®) or erlotinib (TARCEVA®) have been tested without major impact on disease. Though cetuximab was approved by the FDA in 2004 for the treatment of metastatic colon cancer and in 2006 for treatment of head and neck cancer concurrently with radiation therapy, results in other tumors known to have EGFR overexpression, such as pancreatic cancer, were disappointing (Cunningham et al., 2004).

Arming anti-CD3 activated T cells with chemically heteroconjugated anti-CD3×tumor associated antigen (TAA) BiAb converts activated T cells (ATC) into non-major histocompatibility complex (MHC)-restricted serial cytotoxic T lymphocytes (CTL). See Lum & Sen, 2001; Reusch et al., 2006. BiAb arming of ATC via anti-CD3E (OKT3) confers novel anti-tumor-specificity to the T cells regardless of their endogenous MHC-restricted T cell receptor (TCR) specificity. In vitro data demonstrate that BiAb-armed activated T cells (BATs) proliferate, serially kill/lyse tumor cells releasing tumor antigens, secrete interferon-γ (IFN-γ), tumor necrosis factor (TNF-α) and granulocyte-macrophage colony stimulating factor (GM-CSF), as well as chemokines RANTES (CCL5) and MIP-1α (CCL3; Sen et al., 2001; Grabert et al., 2006; Yano et al., 2014), together which can recruit and activate endogenous immune cells (Lee & Margolin, 2011; Sokol & Luster, 2015). The Th₁ cytokine enriched milieu, with antigen cross-presentation by antigen presenting cells (APC), can lead to in situ vaccination and epitope spreading. A major advantage of the BATs platform is the ability to incorporate any anti-TAA antibody, either through chemical heteroconjugation with OKT3 or by recombinant BiAb technology. Based on the expression of their respective targets on solid tumors, chemically heteroconjugated anti-HER2×anti-OKT3 (HER2Bi)-, anti-EGFR×anti-OKT3 (EGFRBi)-, and anti-GD2×anti-OKT3 (GD2Bi)-armed BATs have been developed and are in clinical testing (Lum et al., 2015; Vaishampayan et al., 2015; Yankelevich et al., 2019; Lum et al., 2020b).

Recombinant tetravalent BiAb (rT-BiAb) formats offer several advantages over chemically heteroconjugated BiAbs for arming anti-CD3 activated T cells: (1) they can be produced as homogeneous full length molecules as opposed to the multiple antibody component mixture obtained from chemical heteroconjugation; (2) production costs using standard industry methods are much lower, with higher yields; (3) the overall activity and target range of BATs may be improved; and (4) arming with multiple BiAbs with different specificities can provide enhanced efficacy. We demonstrate herein the production of a novel recombinant tetravalent EGFRBi (rEGFRBi) and the functional activity and clinical potential of rEGFRBi-armed ATC against a broad range of solid tumor cell lines.

### Example 1

### Expression of rEGFRBi

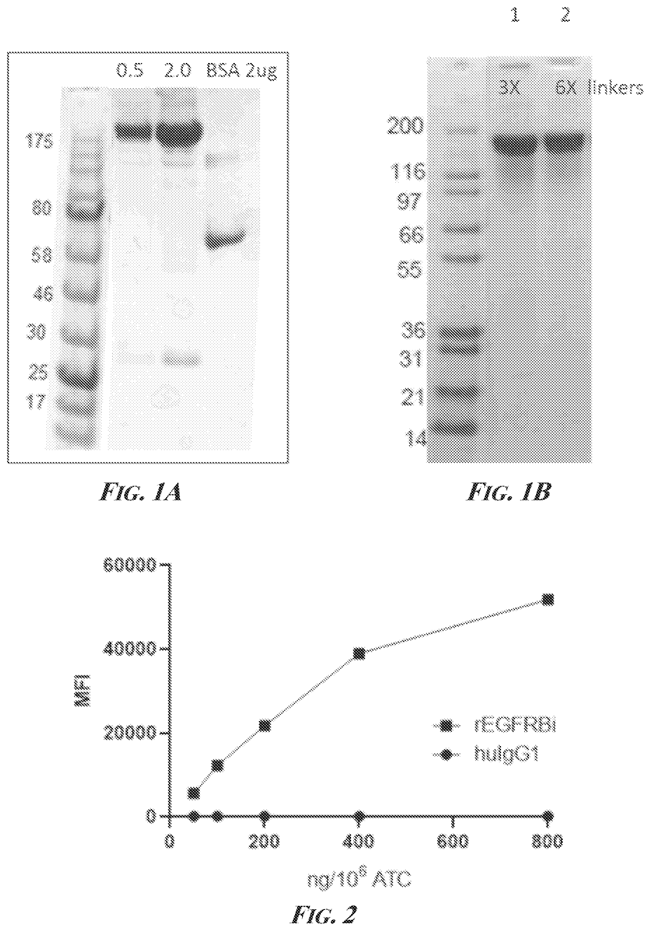

High expressing clones were used to obtain rEGFRBi by Protein A column chromatography purification and characterization by SDS PAGE. rEGFRBi is expressed as a single band of the expected size at a concentration of approximately 300 mg/L in batch-fed culture (see FIGS. 1A and 1B). Clone 1E2 was chosen for functional characterization.

### Example 2

### Binding of rEGFRBi to ATC and EGFR-Expressing Cell Lines

As rEGFRBi comprises an anti-CD3 scFv linked to the heavy chain of an Erbitux-human IgG1 Fc construct, the binding of rEGFRBi to ATC, and rEGFRBi and Erbitux to solid tumor targets was compared using flow cytometry. For binding to ATC, a concentration range of 50 to 800 ng/10⁶ was used to arm 0.5 million cells from a normal donor. The median fluorescence intensity (MFI) of PE-anti-human IgG secondary antibody staining was plotted for the rEGFRBi vs. the same concentration of human IgG1. rEGFRBi showed a linear increase in binding to ATC up to 400 ng/10⁶ ATC and began to plateau at 800 ng/10⁶ (see FIG. 2).

All of the cell lines used in this study express easily detectable levels of EGFR with the exception of MCF-7, which expresses very low levels. rEGFRBi and Erbitux® were incubated with SKBR3, MIA PaCa-2, and MCF-7 cells at between 0.5 to 4 g/mL followed by a PE-anti-human IgG secondary antibody. Both antibodies showed similar MFI at each concentration for all three cell lines (see Table 3). Thus, rEGFRBi demonstrates the ability to attach to ATC and recognize EGFR-expressing target cells.

### Example 3

### rEGFR-BATs are More Cytotoxic than Heteroconjugated HER2Bi-Armed ATC (HER2 BATs) and EGFRBi-Armed ATC (EGFR-BATs) Against their Respective Targets

Several chemically heteroconjugated BiAbs are in clinical trials to treat multiple cancer indications. Preclinical data in support of their activity included in vitro ⁵¹Cr release assays and Luminex Th1 cytokine profiling of co-culture supernatants. The clinical arming dose of these BiAbs is 50 ng/10⁶ ATC. Because the chemically heteroconjugated BiAb reaction is comprised of dimer and multimer BiAb formats as well as unconjugated monomer antibodies, both free OKT3 and BiAb bind to ATC during arming. On a molecular weight basis, the rEGFRBi represents approximately 3-fold more BiAb vs. the heteroconjugated BiAb mixture (depending on particular BiAbs). Therefore, a range of rEGFRBi arming concentrations from between 1 to 400 ng/10⁶ ATC was used in comparison to the standard clinical arming dose of 50 ng/10⁶ ATC (or dose titrations) of heteroconjugated BiAbs. All comparisons among the different BiAbs were made using the same normal donor cells armed under the same conditions.

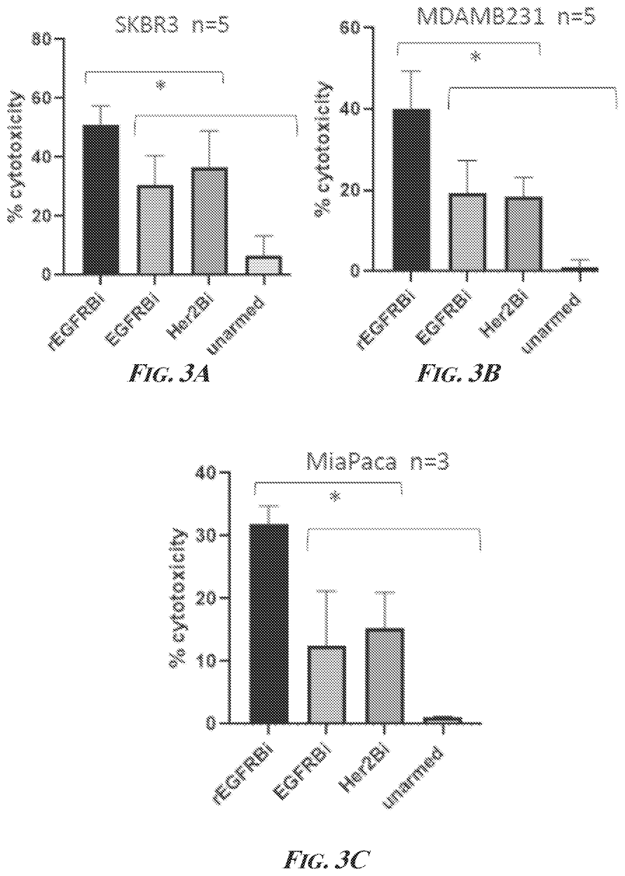

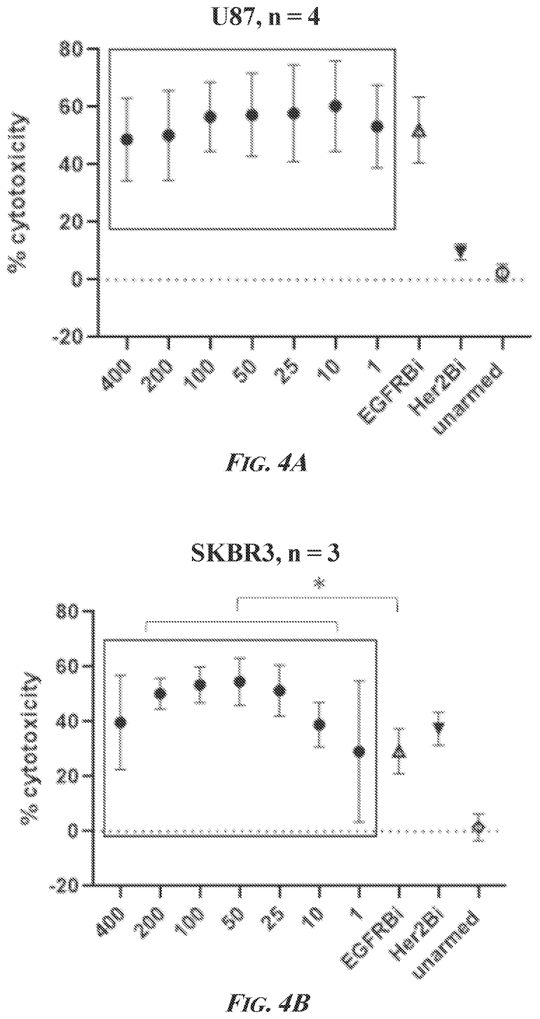

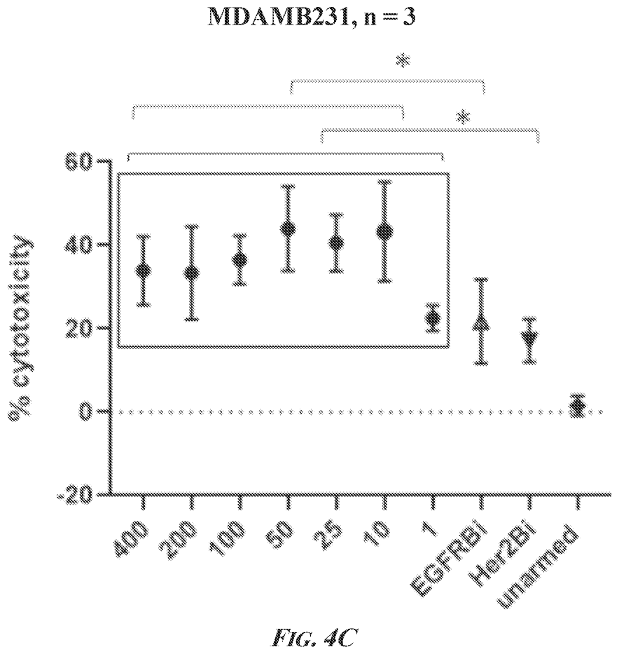

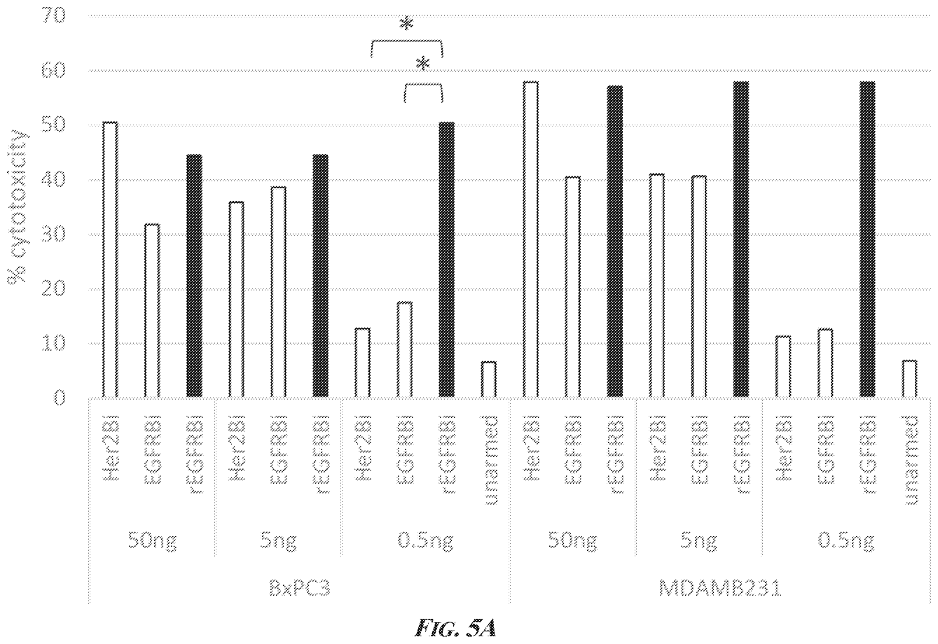

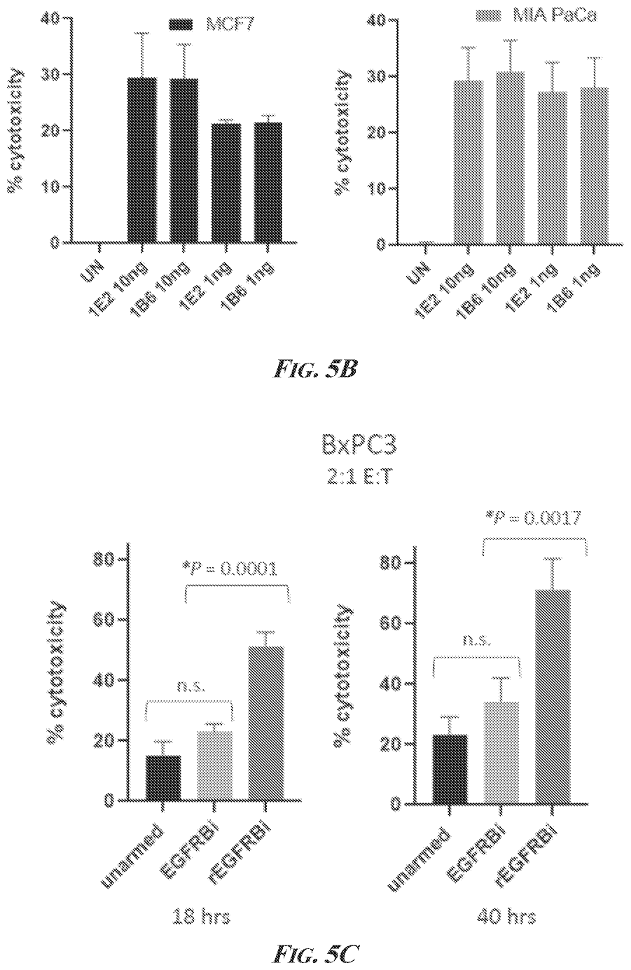

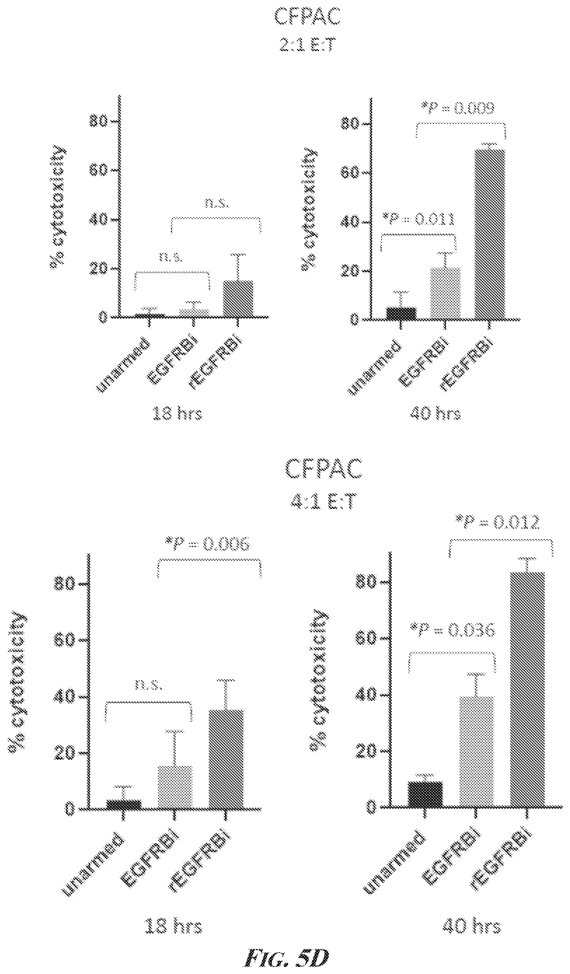

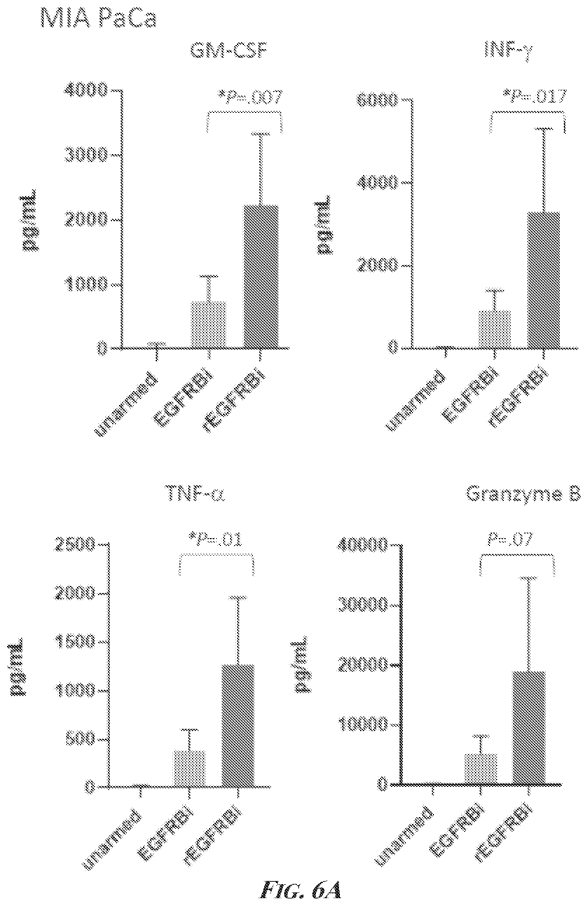

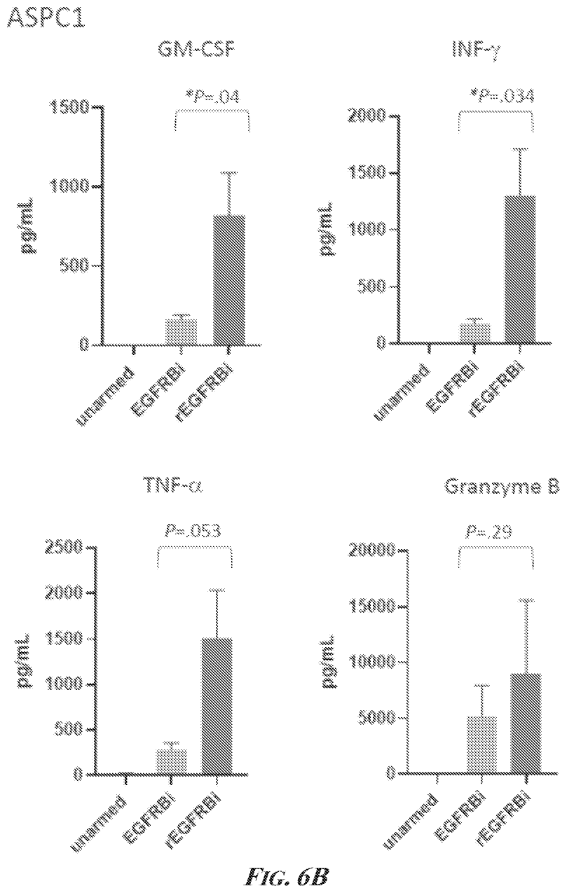

At the E:T of 10:1 and arming concentration of 50 ng/10⁶ ATC, rEGFR BATs showed significantly greater cytotoxicity (between 1.5 to 3 fold) against the breast cancer cell lines SKBR3 and MDAMB-231, and the pancreatic cancer line MIA PaCa-2 using ⁵¹Cr-release assays (see FIGS. 3A-3C). Normal donor ATC were also armed with rEGFRBi with 1, 10, 25, 50, 100, 200 or 400 ng/10⁶ cells and tested against MDA-MB-231, SKBR3 and U87 cells in overnight ⁵¹Cr-release assays. For U87 glioblastoma cells there was no significant difference in cytotoxicity among the different rEGFRBi arming conditions nor between any of the rEGFR-BATs conditions and EGFRBi-BATs armed at 50 ng/10⁶ cells (see FIGS. 4A-4C). And, as demonstrated earlier by Zitron et al., 2013 (Zitron et al., 2013), Her2-BATs did not kill U87 cells. With SKBR3 targets, there was no significant difference among rEGFR-BATs armed between 400 to 10 ng, nor any of those rEGFR-BATs conditions and Her2-BATs. However, rEGFR-BATs armed between 10 and 200 ng were significantly higher than for EGFR-BATs. For MDAMB231, there was no significant difference among rEGFR-BATs between 10 and 100 ng, although all of those were significantly greater than rEGFRBi at 200 and 400 ng. rEGFR-BATs at 25, 50, and 100 ng were significantly higher than at 1 ng, while those at 10, 200 and 400 were not different than 1 ng. However, rEGFRBi-BATs armed from 10-400 ng were significantly higher than for EGFR-BATs, while rEGFR-BATS from 1-400 were significantly higher than for HER2-BATs. When 2 normal donors were armed over a narrower range of 50, 5, and 0.5 ng and tested against BXPC-3 and MDA-MB-231 cells, rEGFR-BATs maintained the same level of killing across all 3 concentrations, while EGFR- and HER2-BATs dropped off at 0.5 ng (see FIG. 5A). The same pattern was seen with a normal donor against MIA PaCa-2 cells (pancreatic cancer). A second clone of rEGFRBi, 1B6, showed similar cytotoxicity to 1E2 against MCF-7 and MIA PaCa cells in a 16-hour ⁵¹Cr-release assay at arming concentrations of 10 ng and 1 ng/10⁶ ATC (see FIG. 5B). Cytotoxicity assays were also performed using the xCELLigence Real Time Cell Analysis (RTCA) system to compare EGFR- vs. rEGFR BATs against pancreatic cancer lines BxPC3 and CFPAC at 2:1 and/or 4:1 E:T which allowed for measurement of cytotoxicity continuously for 40 hours. rEGFR BATs were >2 times as active against both cell lines at both early (18 hours) and late (40 hours) times of incubation (see FIGS. 5C and 5D).

### Example 4

### rEGFR-BATs Release Greater Levels of Th1 Cytokines Against a Broad Range of Cell Lines Derived from Patients with Pancreatic, Breast, and Prostate Cancer

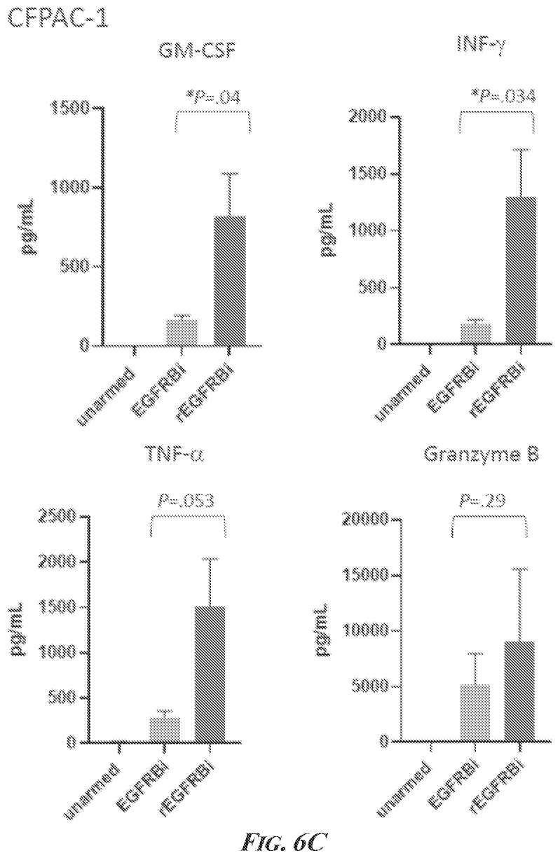

The secretion of Th1 cytokines by rEGFR BATs was compared to that of either EGFR BATs (pancreatic cancer) or HER2 BATs (breast and prostate cancers) at an E:T of 6:1 vs. the respective target cell lines used to treat corresponding patients. In all cases, rEGFR BATs secreted greater amounts of GM-CSF, INF-γ, TNF-α, and Granzyme B (see FIGS. 6A-6E). Table 4 summarizes the relative fold increases in these secretion levels for rEGFR BATs vs. the three chemically heteroconjugated BiAb-armed ATC products. The relative fold increases in Th1 cytokine expression were much higher than for the relative increases in cytotoxicity, ranging between 2.9-7.2 for GM-CSF, 3.6-8.2 for IFN-γ, 5.7 for TNF-α and 1.7-3.6 for granzyme B. These results suggested that the ⁵¹Cr-release assays were a less sensitive indicator of the relative strength of the anti-tumor activity of the different BATs products at least in part due to the significant anti-tumor effects of these factors on tumor growth or survival (Burkholder et al., 2014; Matsuzaki et al., 2015; Shklovskaya et al., 2016; Showalter et al., 2017).

### Example 5

### rEGFR-BATs Release Greater Levels of Th1 Cytokines Over a Range of E:T

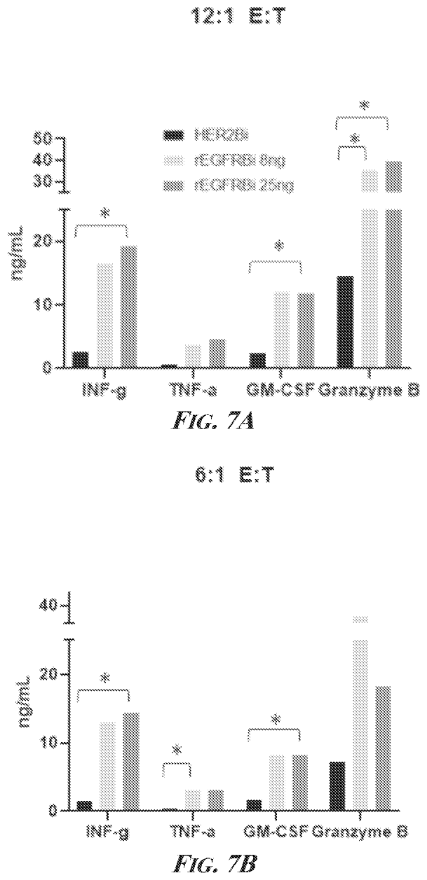

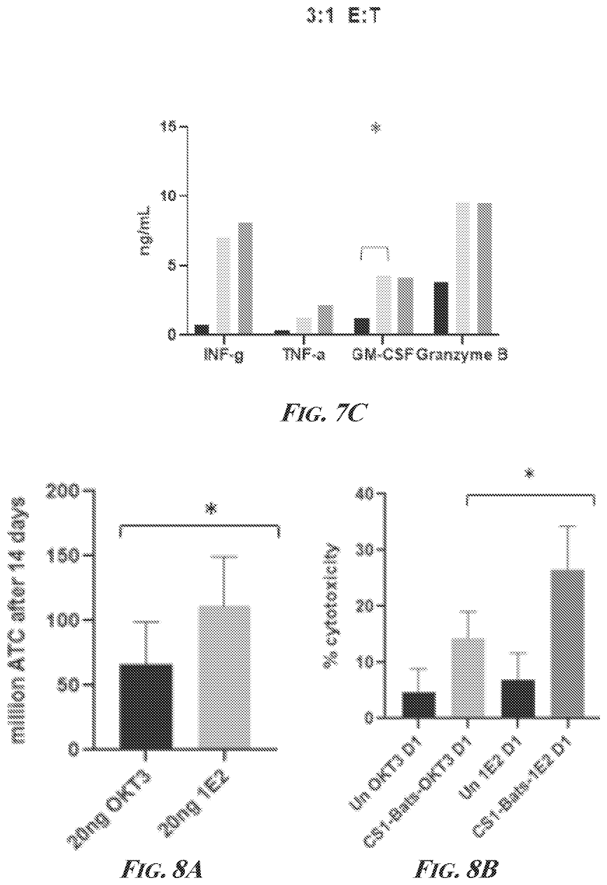

rEGFR-BATs were armed at 8 and 25 ng/million ATC and compared with HER2-BATs (currently used in patients with prostate cancer), against PC3 cells. At E:T's of 12, 6 and 3:1, rEGFR-BATs produce 5-10 fold higher levels of IFN-γ, TNF-α, GM-CSF and IL-2 at each E:T, and more impressively, multifold higher levels at 3:1 than the HER2-BATs at 12:1 (sees FIGS. 7A-7C).

### Example 6

Two Novel Properties of rEGFRBi Greatly Enhance the Therapeutic Potential of BATs Therapy Improved expansion of rEGFRBi- vs. OKT3-activated T cells. PBMC from 4 healthy donors were treated with either OKT3 or rEGFRBi at 20 ng/10⁶ lymphocytes plus 100 U/10⁶ IL2 and expanded with additional IL2 every 48 hours for 14 days. The total number of ATC at 14 days was significantly higher in the rEGFRBi-activated group (mean of +89%, range +33.9-129.4%; see FIG. 8A) indicating that the initial activation of peripheral blood T cells is more efficient for the anti-CD3 variable domain configuration in the rEGFRBi vs. that in the OKT3 monoclonal antibody. The expansion of T cells induced by rEGFRBi activation over a range of 20-400 ng/mL is shown in Table 5.

Superior intrinsic cytotoxicity of rEGFRBi-activated T cells. The ATC activated by OKT3 and rEGFRBi were armed with a BiAb for targeting the CS1 protein expressed by multiple myeloma cells (Lum et al., 2020a) and tested in a flow cytometry-based cytotoxicity assay. Anti-CD3×anti-CS1 BiAB-armed ATC were incubated with ARH77 myeloma cells at 1:1 E:T for 16 hours. Cytotoxicity of the rEGFRBi-activated cells was ˜1.8-fold greater than for OKT3-activated T cells (25.6%±7.8 vs. 14.3±4.7; p<0.05; see FIG. 8B). Thus, rEGFRBi may be able to enhance the total number and baseline cytotoxic potential of patient cells, as well as redirect the more potent effector functions against solid tumors described above.

The use of a T-BiAb in which the first variable sequence recognizes CD3 may also be used in the activation of T cells for the purpose of, e.g., transducing the T cells with transgenes for chimeric antigen receptors. The T-BiAb can be used in addition to the commonly used co-activation beads (binding to CD3 and CD28) or in lieu of those beads with the addition of IL-2, as described above, or in other methods commonly practiced that include anti-CD3 antibodies (Roddie et al., 2019; Wu et al., 2020; Zhang et al, 2020). Similarly, an anti-CD3 T-BiAb can be used to replace anti-CD3 monoclonal antibodies in the culture stage of producing T-regulatory cells (e.g., Chakraborty et al., 2012; Ellis et al, 2012)

Cumulatively, rEGFR-BATs not only killed better at lower arming doses and E:T, they killed every cell line tested. In particular, U87 GBM cells were highly susceptible to rEGFR-BATs, even though they cannot be killed by HER2-BATs. The larger ratios of cytokines released by EGFRBi-BATs engagement is much more pronounced than for cytotoxicity measured by ⁵¹Cr-release assays and may more accurately reflect the relative cytotoxic activity as well as potential for promoting anti-tumor immune responses within the TME. rEGFR BATs may also replace the need for separately targeted BATs (i.e., HER2-, EGFR BATs) against a broad range of solid tumor types.

### Discussion of EXAMPLES 1-6

The data presented herein demonstrated the ability to manufacture full-length, tetravalent rEGFRBi from stably expressing CHO-S cells. rEGFR-BATs were shown to exhibit greater potency than both EGFR- and HER2-BATs in terms of cytotoxicity and/or Th1 cytokine secretion against breast cancer, pancreatic cancer, prostate cancer, and glioblastoma cell lines. They maintained strong levels of killing across a wide range of arming concentrations and release higher levels of cytokines and chemokines even at lower E:T. Bivalency for each variable region specificity was important to maintain attachment of the BiAbs to ATC and optimize activation upon engagement with TAA. Monovalent formats may lead to faster off-rates from the ATC that would reduce or preclude the activity of ex-vivo armed products, and thus require, e.g., continuous infusion of low concentrations of monovalent BiAbs as in the case of blinatumomab. The latter must also take into account systemic interactions with the entire population of circulating T cells, whereas the BATs strategy permitted optimizing the anti-tumor activity of ATC alone. The binding affinity of anti-EGFR in the recombinant BiAb was similar to that of Erbitux® with respect to binding to tumor lines MCF7, MIA PaCa-2, and SKBR3. However, there is a significant difference in the relative binding of EGFRBi and rEGFRBi to ATC. The former is limited by the presence of unconjugated OKT3 in the arming mixture, whereas the latter is capable of increased attachment of at least 20-fold. Functionally, the configuration of OKT3 in rEGFRBi leads to enhanced cytotoxicity and Th1 cytokine secretion by BATs. This improvement is likely due to the shorter spacing between the anti-CD3 and anti-EGFR variable domains in the recombinant vs. the chemical heteroconjugated BiAbs. Upon engagement with tumor cells, the ATC may be brought closer to the tumor cell surface which may lead to more efficient binding of adhesion molecules, TCR crosslinking, or both, that may more efficiently activate T cell responses and/or cell division. Relatively greater clinical responses may therefore be observed with rEGFR-BATs in the form of tumor regression, stronger anti-tumor immune responses, enhanced sensitivity to chemotherapy, and overall survival.

Whereas a closer juxtaposition of the anti-CD3 and anti-EGFR variable regions improves the cytotoxicity and cytokine release of BATs, positioning of the OKT3 scFv to the N-terminus of a full length antibody increases the distance between its bivalent domains vs. their relative position in OKT3, the result of which enhances the ability of rEGFRBi to activate and expand peripheral blood T cells. The latter results in a significantly greater expansion of the total number of ATC and the enhanced cytotoxic potential of those cells.

Clinical trials of HER2-, EGFR- and GD2-BATs armed with chemical heteroconjugated BiAbs have demonstrated the safety and feasibility of multiple BATs infusions for a variety of solid tumors (Lum et al., 2015; Vaishampayan et al., 2015). BATs are produced using BiAbs to arm ex vivo expanded ATCs, which creates an “army” of non-MHC-restricted serial killers. Unlike CAR-T cells, BATs are non-transgenic and are thus self-limiting due to the cell-surface attachment of BiAbs; the concentration of BiAbs decreases as the cells divide and eventually lose activity against their target. BATs are therefore much safer than CAR-T, TCR-T, or CAR-NK cells because they are not transgenic. More important, they have not been associated with CRS even at multiple doses of up to 40 billion/cells per infusion. Multiple infusions of rEGFR BATs are likely to increase overall cytotoxicity and in situ immunization vs. a single infusion of cells due to accumulated benefits from “waves” of tumor engagement. The latter may also provide immunosensitization to overcome chemoresistance and immune suppression.

Immune evaluations in BATs clinical trials demonstrating the induction of patient cellular and humoral anti-tumor responses, and the potential for increased patient survival vs. standard of care, support the concept of BATs providing direct anti-tumor effects as well as modifying the TME to promote an endogenous anti-tumor immune response upon release of TAA and Th1 cytokines (Lum et al., 2015; Thakur et al., 2018). Therefore, strategies that increase the potency of a direct anti-tumor response (e.g., cytotoxicity and expanded cellular and humoral responses) may be further improved by both directly and indirectly downregulating other cells within the TME that inhibit the development of anti-tumor responses and promote tumor growth and metastasis. For example, T regulatory cells and myeloid suppressor cells dampen both cellular and humoral anti-tumor responses; endothelial cells, macrophages, mesenchymal stem cells and stromal cells interact with tumor cells to promote tumor cell growth and/or metastases. While current T cell approaches do not generally consider such mechanisms, evidence for EGFR expression in “accessory” cell types within the TME makes them potentially targetable by EGFR-BATs. We have previously shown that EGFR-BATs co-cultured with MIA PaCa-2 pancreatic cells in a 3D co-culture model of PBMC decreased the differentiation of MDSC in the presence of Th1 cytokines (Thakur et al., 2013).

The higher activity of rEGFR-BATs also has the potential to increase toxicity due to either direct cytotoxicity against tumor tissue and other cells of the TME, or the release of Th1 cytokines. The ability to arm ATC over a wide range of concentrations provides an important mechanism to control potency, and because BATs are inherently self-limiting, further ways to regulate their activity include modifying the number of BATs per infusion and the overall number and frequency of infusions. In this manner, BATs can be administered more like a conventional drug vs. a long-lived replicating, gene-modified cell.

The expression of EGFR on a broad range of solid tumors, the role of EGFR signaling in cancer stem cell biology, the upregulation of EGFR on Tregs in cancer patients, and its expression on other tumor promoting cells of the TME, make the rEGFR-BATs product suitable for many solid tumor indications. If broadly effective, they would preclude the need for extensive preclinical screening and safety testing of novel anti-TAA antibodies, some of which have caused serious side effects in Phase 1 studies as CART cells. The improved potency demonstrated in this study supports the testing of rEGFR-BATs in clinical trials for indications including pancreatic, breast, ovarian and prostate cancers, glioblastoma and others.

### Example 7

### OKT3-HER2 T-BiAbs

The variable light chain and heavy chains of humanized OKT3 were also assembled as V_{L}-(G4S)₆-V_{H} chain and V_{L}-(G4S)₃-V_{H} chain scFvs and fused to the heavy chain of anti-HER2 heavy chain of Herceptin with the linker GGGGSGGGGSGGGTSGGGGSGGGGS (SEQ ID NO: 10). The heavy chain fusion genes and light chain expression vectors were co-transfected into 293 cells for transient transfection and purification (FIG. 1B). Both T-BiAbs expressed as full-length antibodies. However, the version with the (G4S)₆ linker scFv expressed at 2-fold higher levels under identical transfection conditions.

The linker sequences used for the scFvs of the OKT3-based T-BiAbs and the N-terminus linkers are summarized in Table 6.

### Example 8

### Other OKT3 scFv-Based T-BiAbs for Cancer

The general approach of arming ATC via OKT3 scFv-based T-BiAbs can be applied to fusion constructs with anti-tumor associated antigen antibodies, similar to those used in chemically heteroconjugated BiAbs, recombinant BiAbs, and the targeting scFvs of CAR T cells. These targets include, but are not limited to, MAG-1 (North et al., 2011), CD19, CD20, CD22, CD30, CD33, CS1/SLAMF7, B cell maturation antigen (BCMA), CD38, CD123, HER2, HER3, and others (see Table 7; from Strohl & Naso, 2019).

The strong activity of rEGFR BATs over a broad arming range provides the option to multiply-arm ATC with 2 or more different targeting BiAbs without saturating the available T cell receptors or non-specifically activating armed T cells. This allows targeting of the same cell type via multiple surface antigens, and/or targeting different cell types with the same or different BiAbs. T-BiAbs that recognize surface antigens other than CD3 or the T cell receptor may be added to improve the functional activation of T cells or to block inhibitory signals. Examples include anti-EGFR plus anti-CS-1 or BCMA BiAbs for multiple myeloma, anti-EGFR plus anti-CD20 or CD19 for chronic lymphocytic leukemia, among others.

Sequence 1: VS327 = OKT3-EGFR V_{H} Xba1-Nhe1 cloning cassette nucleotide sequence for
BO VECTOR-059;
SEQ ID NO: 1
tctagaCCACCATGAAGTTGCCTGTTAGGCTGTTGGTGCTGATGTTCTGGATTCCTGCTTCCA
GCAGTGACATCCAAATGACCCAGAGCCCCAGTAGCCTGAGTGCCAGCGTGGGCGACAGG
GTGACCATCACCTGCAGTGCAAGCAGCAGCGTGAGCTACATGAACTGGTATCAACAGAC
CCCAGGCAAGGCGCCCAAAAGGTGGATCTACGACACCAGCAAGCTGGCGAGCGGTGTGC
CCAGCAGGTTTTCCGGTAGTGGGTCAGGCACCGACTACACCTTCACTATCAGCAGCCTGC
AGCCCGAGGACATCGCCACCTATTACTGCCAGCAATGGAGCAGCAACCCCTTCACCTTCG
GACAGGGCACGAAGCTGCAGATCACGAGGGGTGGAGGCGGTAGCGGTGGGGGAGGCTC
AGGGGGCGGAGGCAGTGGCGGTGGCGGGAGCGGGGGTGGTGGTTCAGGAGGAGGTGGC
AGTCAGGTGCAGCTGGTGCAGAGCGGAGGGGGTGTGGTACAGCCCGGCAGGAGCTTGAG
GCTGAGCTGCAAGGCCAGCGGCTATACCTTTACGAGGTACACCATGCACTGGGTGAGGCA
AGCGCCTGGTAAGGGCCTGGAGTGGATCGGGTACATCAACCCTAGCAGGGGCTACACTA
ACTACAACCAGAAGTTCAAAGACAGATTCACGATTAGCACAGATAAGAGCAAGTCTACC
GCTTTCCTCCAGATGGACAGCCTTAGGCCTGAGGACACCGCCGTGTACTACTGCGCAAGG
TACTACGACGACCACTACTGTCTTGACTATTGGGGGCAGGGGACTCCCGTGACCGTGAGC
AGCGGTGGAGGAGGTAGTGGAGGAGGCGGAAGTGGTGGTGGAACATCGGGAGGCGGAG
GTTCAGGAGGTGGAGGCTCTCAGGTGCAGCTGAAGCAGTCAGGACCTGGCCTAGTGCAG
CCCTCACAGAGCCTGTCCATCACCTGCACAGTCTCTGGTTTCTCATTAACTAaCTATGGTG
TACACTGGGTTCGCCAGTCTCCAGGAAAGGGTCTGGAGTGGCTGGGAGTGATATGGAGTG
GTGGAAaCACAGACTATAATaCAcCTTTCAcATCCAGACTGAGCATCAaCAAGGACAATTCC
AAGAGCCAAGTTTTCTTTAAAATGAACAGTCTGCAAtCTAATGACACAGCCATATATTACT
GTGCCAGAgcactTacTtactacgatTacgaatTTgcttactggggccaagggactctggtcactgtctctgcagctagc
NOTE: the 6 nucleotide XbaI recognition sequence (tctaga) at the 5′ end and the 6
nucleotide NheI recognition sequence (gctagc) at the 3′ end are both underlined above.
VS327 = OKT3-EGFR V_{H} Xba-Nhe1 amino acid sequence encoded by the cloning cassette for
BO VECTOR-059 of SEQ ID NO: 1:
SEQ ID NO: 2
MKLPVRLLVLMFWIPASSSDIQMTQSPSSLSASVGDRVTITCSASSSVSYMNWYQQTPGKAPK
RWIYDTSKLASGVPSRFSGSGSGTDYTFTISSLQPEDIATYYCQQWSSNPFTFGQGTKLQITRG
GGGSGGGGSGGGGSGGGGSGGGGSGGGGSQVQLVQSGGGVVQPGRSLRLSCKASGYTFTR
YTMHWVRQAPGKGLEWIGYINPSRGYTNYNQKFKDRFTISTDKSKSTAFLQMDSLRPEDTAV
YYCARYYDDHYCLDYWGQGTPVTVSSGGGGSGGGGSGGGTSGGGGGGGGSQVQLKQSGP
GLVQPSQSLSITCTVSGFSLTNYGVHWVRQSPGKGLEWLGVIWSGGNTDYNTPFTSRLSINKD
NSKSQVFFKMNSLQSNDTAIYYCARALTYYDYEFAYWGQGTLVTVSAAS
NOTE: the CDRs, SEQ ID NOs: 14-22, are underlined above.
Sequence 2: VS328 = OKT3-EGFR Vx Xba1-BsiW1-BamH1 cloning cassette nucleotide
sequence in BO VECTOR-042; Sequence 2;
SEQ ID NO: 3
tctagaGCCGCCACCATGAAGTTGCCTGTTAGGCTGTTGGTGCTGATGTTCTGGATTCCTGCT
TCCAGCAGTGACATCtTGCTGACaCAaTCTCCAGtCATCCTGTCTGTGAGTCCAGGAGAAAG
AGTCAGTTTCTCCTGCAGGGCCAGTCAGAGCATTGGCaCAAacatcCACTGGTATCAaCAgAG
AACAAATGGTTCTCCAAGGCTTCTCATAAAGTATGCTTCTGAGTCTATtTCTGGGATCCCTT
CCAGGTTTAGTGGCAGTGGATCAGGGACAGAcTTTACTCTTAGCATCAACAGTGTGGAGT
CTGAAGATaTTGCAGATTATTACTGTCAACAAAaTAATAacTGGCCtacaACGTTCGGTgcaGGa
ACgAagcTGGAactcAAAcgtacggtggctgcaccatctgtcttcatcttcccgccatctgatgagcagttgaaatctggaactg
cctctgttgtgtgcctgctgaataacttctatcccagagaggccaaagtacagtggaaggtggataacgccctccaatcgggtaa
ctcccaggagagtgtcacagagcaggacagcaaggacagcacctacagcctcagcagcaccctgacgctgagcaaagcagactac
gagaaacacaaagtctacgcctgcgaagtcacccatcagggcctgagctcgcccgtcacaaagagcttcaacaggggagagtgtt
agagggagaagtgcccggatcc
NOTE: the 6 nucleotide XbaI recognition sequence (tctaga) at the 5′ end, the central
6 nucleotide BsiW1 recognition sequence (cgtacg), and the 6 nucleotide BamHI
recognition sequence (ggatcc) at the 3′ end are underlined above.
VS328 = OKT3-EGFR Vκ expression cassette amino acid sequence encoded by the cloning
cassette for BO VECTOR-042 of SEQ ID NO: 3;
SEQ ID NO: 4
MKLPVRLLVLMFWIPASSSDILLTQSPVILSVSPGERVSFSCRASQSIGTNIHWYQQRTNGSPRL
LIKYASESISGIPSRFSGSGSGTDFTLSINSVESEDIADYYCQQNNNWPTTFGAGTKLELKRTVA
APSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC*
NOTE: the three CDRs, SEQ ID NOs: 23-25, are underlined above.

### Example 9

### CD34 scFv-Linked T-BiAbs

Diabetes mellitus (DM) is a major health problem worldwide, affecting over 10% of the US population and posing a major risk factor for acute myocardial infarction (AMI) mortality and hospitalizations (Jacoby & Nesto, 1992; Aronson et al., 1997; Nesto & Zarich, 1998; Luo et al., 2013). Women with late DM diagnosis are at even greater risk than men. With overwhelming evidence linking diabetic vascular complications to endothelial dysfunction, new therapeutic strategies are required to treat high-risk DM patients, particularly those suffering from AMI (Jarajapu & Grant, 2010; Roche & Wang, 2013).

DM impairs multiple endothelial repair mechanisms. Since the observations of Asahara et al., 1997 describing the role of endothelial progenitor cells (EPCs) in postnatal neovascularization, extensive efforts have been made to further characterize their mechanisms of repair and to develop cell therapies for ischemia and cardiovascular diseases (Asahara et al., 2011). Effective repair requires EPC mobilization, survival in peripheral blood, and recruitment/attachment to damaged tissue. However, DM significantly inhibits both the quantitative and qualitative aspects of EPC function, with evidence for reduced number of circulating EPC, inefficient migration, altered cytokine profiles and lack of regenerative activity (Fadini et al., 2005; Jarajapu & Grant, 2010; Caballero et al., 2007). Diabetic autonomic neuropathy (DAN) is believed to further reduce the number of circulating CD34⁺ cells by 40% vs. patients without DAN (Albiero et al., 2014). Thus, despite the promising clinical results of various therapeutic stem cell (SC) trials, patients with DM are less likely to benefit from such approaches due to inherent deficiencies in their SC populations.

Cell source and reduced migratory activity of circulating progenitor cells are linked to weaker response to SC therapy after AMI. In a dose escalation study of intracoronary (IC)-injected CD34⁺ bone marrow cells (PreSERVE trial), Neostem observed that AMI patients who received over 20 million bone marrow-derived CD34 cells (19% of cell-treated patients) had a significant increase in left ventricular ejection fraction (LVEF) at 6 months, while those receiving less than 20 million cells showed no difference vs. controls (Neostem, 2015). In an analysis of the TIME (Traverse et al., 2012) and Late TIME (Traverse et al., 2011) randomized trials of IC injected total bone marrow cells, Cogle et al., 2014 noted that only those patients whose bone marrow CD34⁺ cell numbers were outside the normal range (>2 standard deviations higher or 4.3% of treated patients) showed an increase in LVEF, which was on the level of improvement in the PreSERVE trial. Moreover, 5 out of those 9 patients had received placebo. One explanation for the difference between the TIME and PreSERVE trials is in the relative retention of whole bone marrow vs. CD34-enriched cells. Whereas only 1.3%-2.6% of ¹⁸F-FDG-labeled unselected BM cells were retained in infarcted myocardium after IC infusion, 14%-39% of CD34-enriched cells were found primarily in the infarct border zone (Hofmann et al., 2005). The effects of relative retention were likely exaggerated by greater numbers of CD34 cells being injected. In animal models, human cord blood cells are reparative for myocardial infarction by i.v. injection, whereas bone marrow or peripheral blood derived cells must be IM injected or armed with bispecific antibodies if injected i.v. to have an effect on infarct size, functional output and vasculogenesis (Lee et al., 2007; Zhao et al., 2008; Yu et al., 2015). Similarly, cord blood CD34 cells show the greatest homing potential to SDF-1 followed by bone marrow and peripheral blood sources (Aiuti et al., 1997).

In an analysis of 172 AMI patients, Fortunato et al., 2013 found that patients who suffered a major adverse event (death, recurrent AMI, or new-onset AMI) had a significantly higher percentage of SCs non-migrating to vehicle (25.65% vs. 16.28% of CD34⁺/CD45⁺/CD133⁺/CXCR4⁺). Adverse event patients also displayed higher levels of serum SDF-1, an effect shown by Subramanian et al., 2014 to be associated with heart failure and all-cause mortality (although not with AMI) in an analysis of 3350 Framingham Heart Study participants.

Methods are needed to improve the retention and function of aged and diseased SCs in patients with AMI. The evidence for diminished SC capacity in older and diabetic patients, plus the inefficiency of SC homing to ischemic myocardium, calls for methods to enhance SC retention to at least meet the minimum threshold for tissue revascularization and prevention of remodeling. Localization through IC or IV injection alone is relatively inefficient, and not likely to help with more compromised cell types.

Bispecific antibodies (BiAb) greatly increase retention and therapeutic efficacy of SC in infarcted myocardium. We have demonstrated a broad range of therapeutic effects from BiAb-targeting of CD34⁺ SCs to ischemic myocardium (Yu et al., 2015). Animals that received peripheral blood (PB) CD34⁺ armed with anti-CD45×anti-myosin light chain (MLC) BiAbs, but not those receiving unarmed cells, achieved the following benefits: smaller infarct size, improvements in a broad range of echocardiographic parameters, normalization of conductance, reduced apoptosis, and enhanced angiogenesis. These experiments demonstrate the ability of BiAbs to produce significant therapeutic effects from an otherwise suboptimal dose of cells. In a similar fashion, we propose to improve the potency of suboptimal SC found in DM and other high-risk patients with AMI.

BiAb-targeting of SC changes the current practice of cell therapy for AMI and regenerative medicine. The current treatment paradigm for AMI is to extract bone marrow cells, obtain total mononuclear or enriched CD34⁺ cells and infuse them into the heart by intracoronary (IC) injection. This method obtains a limited number of progenitor cells, and/or includes a mixture of differentiated cells that might be deleterious to tissue repair, e.g., by increasing inflammation and/or fibrosis. It involves catheter-mediated delivery of cells with limited overall retention in the infarcted tissue (Hoffman et al., 2005). In comparison, BiAb-arming of purified SCs from mobilized peripheral blood greatly increases the local dose of SCs and renders them all capable of binding to the damaged tissue. It establishes a non-invasive means of SC delivery as well as facilitates multiple dosing. BiAb-armed SC delivery results in more uniform improvement of cardiac function vs. intramuscularly injected (IM) cells (Yu et al., 2015). Our preclinical studies indicate that BiAb-armed SCs greatly improve the efficiency of cell delivery and tissue repair vs. unarmed cells and may overcome the limited and inconsistent effects thus far observed in patients with suboptimal SC. The binding specificities of the BiAb can be easily manipulated to accommodate a wide range of SC types and diseased tissues to broadly address the needs of regenerative medicine. Enhanced tissue retention by BiAbs has been independently confirmed by Malecki et al., 2013 using combinations of anti-myosin light chain and anti-CD34, -CD117 and -CD133 antibodies.

Anti-CD34×anti-myosin light chain (MLCBi) is a novel tetravalent bispecific antibody with dual functions for single step arming and isolation of SC ready for injection. MLCBi is a recombinant bispecific antibody that incorporates bivalent antibody binding domains for the anti-SC (anti-CD34) and anti-tissue (anti-MLC) binding components. The principles for this approach have been established separately for each of the dual functions: first, the CliniMACS CD34 cell isolation system (Miltenyi Biotech, GmbH) for isolating hematopoietic SC (HSC) has been FDA approved to enrich CD34 cells for clinical applications. It achieves an average of 96% purity by high gradient magnetic separation of cells bound to an anti-CD34 antibody conjugated to superparamagnetic microbeads (Richel et al., 2000). Such cells were safely administered to 60 breast cancer patients following high-dose chemotherapy leading to safe, long-term engraftment. For commercial use, we intend to follow this approach by conjugating magnetic beads to the BiAb that could be used in the CliniMACS or other magnetic separation system. Second, the anti-MLC portion of the BiAb provides the targeting specificity for the cells without having to perform any additional manipulations. Thus, patients would undergo a leukapheresis followed by an automated process to separate CD34 cells (in approximately 45 minutes) that could be directly infused into the patient or frozen, if necessary, for subsequent use.

BiAb-armed SC improve multiple parameters of heart function and reduce infarct size. We have tested BiAb-armed CD34 cells in three different animal models of myocardial ischemia, all of which have demonstrated significantly higher activity of BiAb-armed cells vs. unarmed cells or phosphate buffered saline (PBS) controls: 1) a rat ischemia/reperfusion model (Lee et al., 2007; Yu et al., 2015); a mouse total left anterior descending artery (LAD) occlusion model with Langendorff isolated heart perfusion preparation (Zhao et al., 2008); and a mouse total LAD occlusion model with time course echocardiography (preliminary data herein). Intravenous (IV) delivery of BiAb-armed CD34 cells leads to greater cellular retention in the infarct; increases in left ventricular (LV) ejection fraction (EF), diastolic and systolic pressures, fractional shortening, angiogenesis, and systolic anterior septal wall thickness; a decrease in infarcted tissue; less severe increases in end diastolic and end systolic volumes; and more uniform conduction than unarmed cells or PBS alone.

Targeted delivery of CD34⁺ cells will overcome the subthreshold levels of dysfunctional SC in DM and/or other high-risk AMI patients with low numbers of SDF-1 migrating SC. A large body of clinical evidence supports that BMSC therapy improves cardiac performance, limits the size of infarct damage, and reduces major adverse clinical events in patients with AMI (Abdel-Latif et al., 2007; Zimmet et al., 2012; Schachinger et al., 2006a; Schachinger et al., 2006b; Neostem, 2015). However, the highest-risk patients are unlikely to benefit from autologous bone marrow or PB CD34⁺ cells. This proposal is designed to overcome the insufficient number of competent SC retained in the infarct of DM and other high-risk AMI patients.

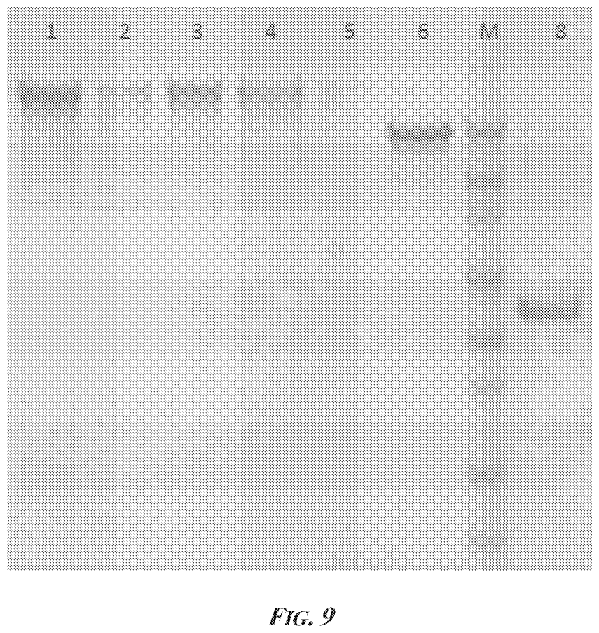

Construction of expression vectors for anti-CD34×MLC BiAbs. Cloned variable regions for four anti-CD34 antibodies were converted to scFvs using TransTarget Inc.'s (TTI) proprietary linker sequences and fused to the N-terminus of the heavy chain for the anti-MLC antibody, clone B12, whose heavy and light chain variable regions had been previously fused to human IgG1 Fc and human light chain constant regions, respectively (Tables 3 and 4). Expression vectors containing the different MLCBi fusion proteins were co-transfected with the complementary MLC light chain expression vector into 293S cells and the BiAbs isolated from tissue culture supernatants using Protein G column chromatography. Samples were run in SDS PAGE gels to determine the MW, degree of full-length expression and/or fragmentation, and relative yield per number of transfected cells (FIG. 9). All 4 BiAbs produced high percentages of full-length antibodies, although construct 387 containing the A30 clone for anti-CD34 was a very weak expressing vector under these transient conditions.

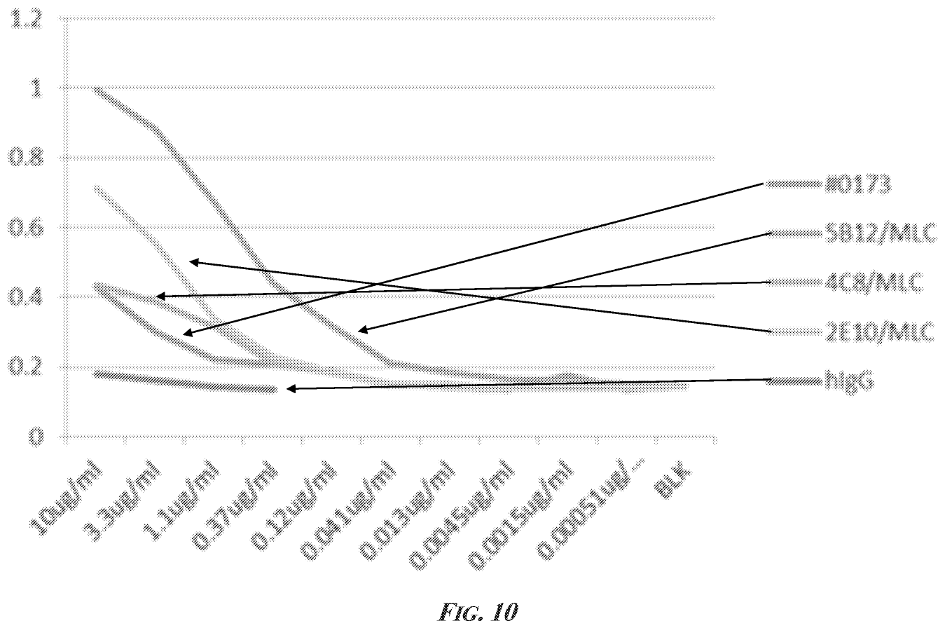

Comparative binding of the MLCBi candidates to porcine cardiac myosin (PCM). The 3 best expressing full-length MLCBi clones (380, 385, and 386) were tested for binding to PCM by ELISA. Briefly, 96 well plates were coated with 10 g/mL PCM overnight at 4° C., followed by incubation of 3-fold dilutions of each BiAb starting at 10 g/mL for 1 hour at 37° C. Bound BiAbs were detected with horse radish peroxidase (HRP)-conjugated anti-huIgG. Clone 380 (clones 5B12×B12) showed the highest absolute binding over the tested range, which was significantly higher than a monovalent BiAb directed against MLC and human CD45 (#0173) (FIG. 10).

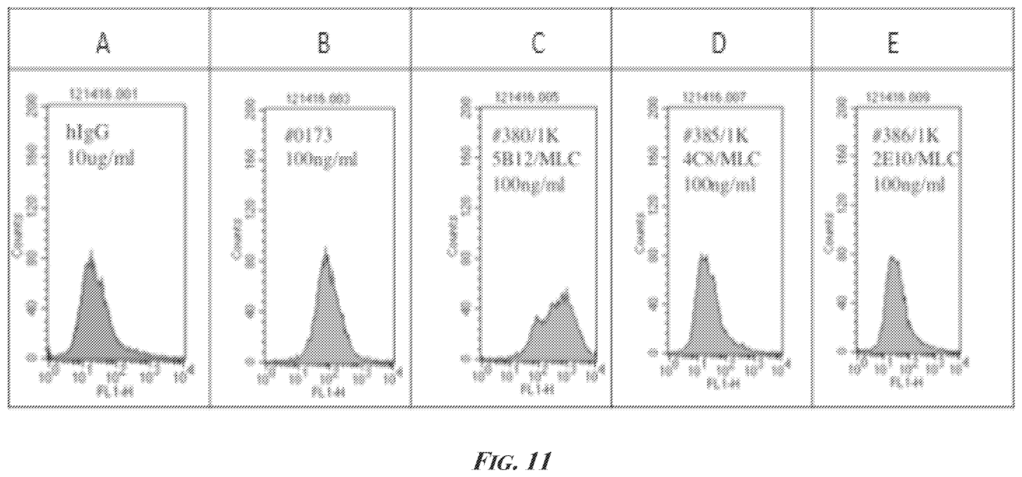

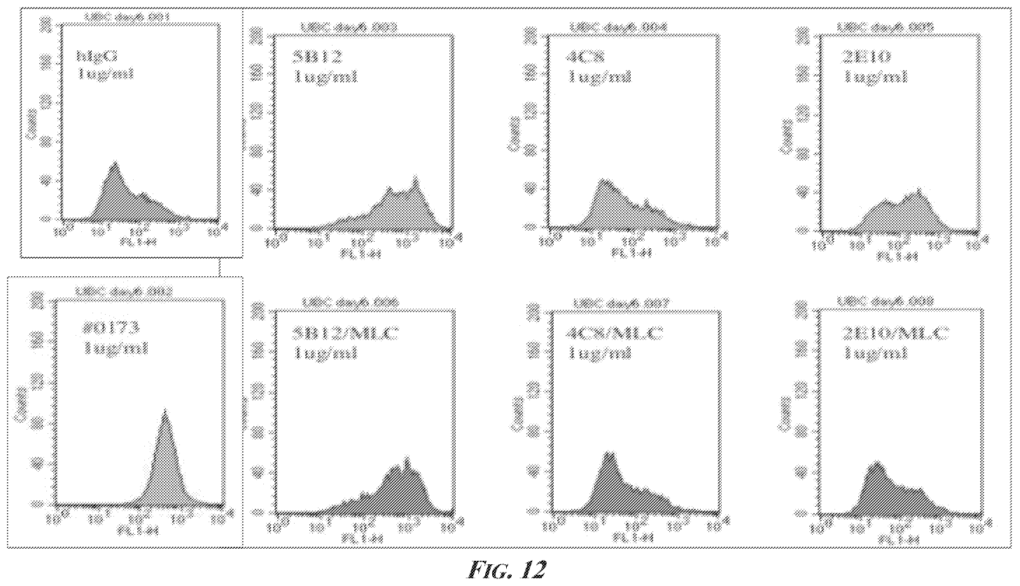

Comparative binding of the MLCBi clones to CD34⁺ KG-1 (acute myeloblastic leukemia [AML] cell line) and umbilical cord blood (CB) CD34⁺ cells. Umbilical cord blood samples were obtained from the Umbilical Cord Blood Collection Center at U.C. Davis by overnight delivery. CD34⁺ cells were isolated using Miltenyi magnetic beads and placed into culture for expansion using methods described in Wagner et al., 2016. KG-1 and CD34⁺ CB cells were stained with varying concentrations of the 3 MLCBiAbs (FIGS. 11 and 12). Clone 380 containing the 5B12 anti-CD34 binding domains showed the greatest level of staining vs. 385 and 386 on both cell types and was therefore selected for use in animal models based on the combined properties of (a) full-length and overall expression, (b) binding to PCM and (c) binding to CD34⁺ cells. The monovalent BiAb, 0173, showed greater binding to CD34⁺ CB cells despite its monovalent format, presumably due to the greater expression level of CD45 vs. CD34 on those cells.

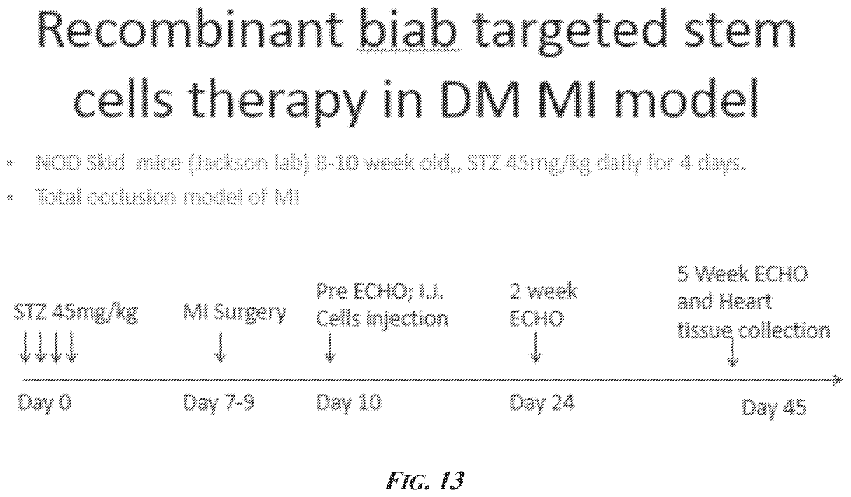

Effect of MLCBi-armed CD34⁺ CB cells on ejection fraction (EF) and glucose tolerance. Groups of 5 NOD/SCID male and female mice were treated with streptozotocin (STZ) to induce hyperglycemia, followed by left anterior descending (LAD) artery total occlusion. Two days post-induction of myocardial infarction, 2 million armed or unarmed CD34⁺ expanded CB cells were given IV via tail vein. Mice were monitored by echocardiography for EF at 2 weeks and 5 weeks post treatment. Animals were tested for blood glucose levels determined at the same time points (FIG. 13). Xing et al., 2009 previously reported the ability of umbilical cord blood (CB) cells injected IV into rats with myocardial infarcts to improve EF at 4 weeks post-injection vs. animals that did not receive cells. The objective of this project was to demonstrate improved efficacy of CB CD34⁺ cells by the addition of a targeting BiAb. Therefore, unarmed CD34⁺ CB cells were used as the comparative control group vs. animals that received no cell infusions.

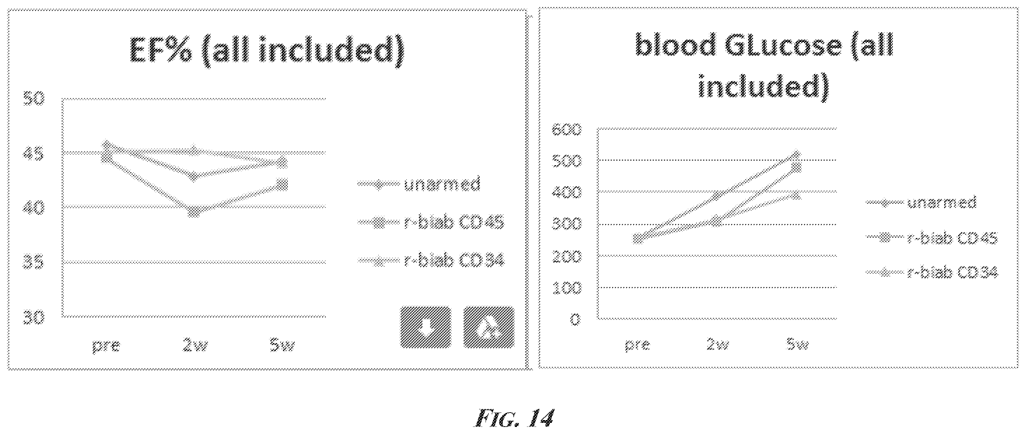

When results were analyzed as a combination of both male and female treated animals, there were no significant differences in ejection fraction or glucose tolerance at 5 weeks post-treatment (FIG. 14). There was also no expected decrease in ejection fraction in the unarmed CD34⁺ cell group. Thus, one possible explanation for our results is that unarmed CB CD34⁺ cells have significant therapeutic benefit that is not capable of further improvement due to tissue targeting under these conditions.

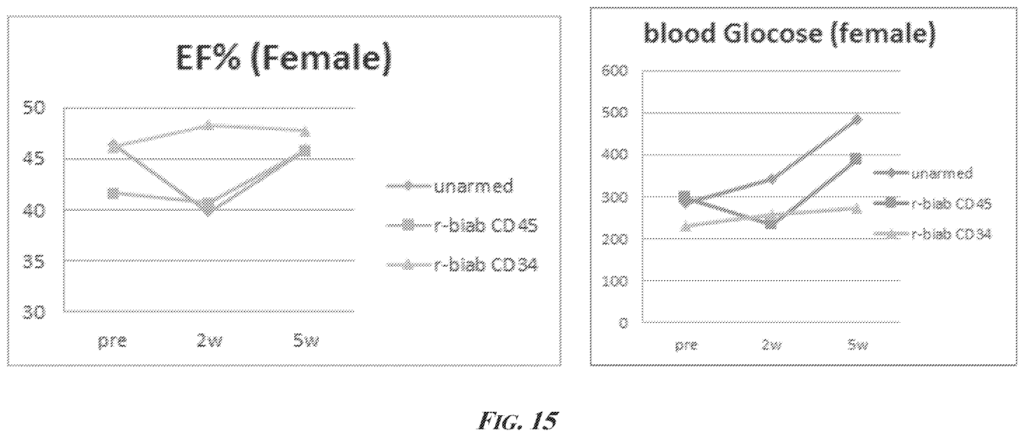

The most interesting observations relate to the potential benefit of CD34 MLCBi-armed CD34+CB cells in female vs. male animals. In female mice, EF is maintained at the same starting level at both 2 wk and 5 wks post-treatment, whereas in male mice the EF at 2 weeks is lower for both unarmed cells and those armed with the anti-CD45×anti-MLC BiAb 0173 (FIG. 15). More importantly, the ability of female mice to maintain normal levels of glucose is conferred only by the CD34×MLC BiAb (380)-armed cells (FIG. 15). Due to the fact that STZ-treated female rats show a relative increase of MLCB protein in skeletal tissue vs. non-STZ-treated rats (Choi et al., 2013), an intriguing association may exist between expression of MLC in females with diabetes and the ability of MLC-targeted CD34⁺ stem cells to promote the repair of damaged islet β-cells. Further experiments are needed to confirm the preferential benefit of targeted stem cells in females, and the potential role of MLC and MLC kinase in the SC repair processes.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Abdel-Latif et al. (2007) Adult bone marrow-derived cells for cardia repair. Arch Intern Med 167:989-997.
Aiuti et al. (1997) The chemokine SDF-1 is a chemoattractant for human CD34+ hematopoietic progenitor cells and provides a new mechanism to explain the mobilization of CD34+ progenitors to peripheral blood. J Exp Med 185(1):111-120.
Albiero et al. (2014) Diabetes causes bone marrow autonomic neuropathy and impairs stem cell mobilization via dysregulated p66Shc and Sirt1. Diabetes 63:1353-1365.
Altschul et al. (1990a) Basic local alignment search tool. J Mol Biol 215:403-410.
Altschul et al. (1990b) Protein database searches for multiple alignments. Proc Natl Acad Sci USA 87:14:5509-5513.
Altschul et al. (1997) Gapped BLAST and PSI-BLAST: a new generation of protein database search programs. Nucleic Acids Res 25:3389-3402.
Aronson et al. (1997) Mechanisms determining course and outcome of diabetic patients who have had acute myocardial infarction. Ann Intern Med 126:296-306.
Asahara et al. (1997) Isolation of putative progenitor endothelial cells for angiogenesis. Science 275:964-967.
Asahara et al. (2011) Concise review: circulating endothelial progenitor cells for vascular medicine. Stem Cells 29:1650-1655.
Barbas (1995) Synthetic human antibodies. Nature Medicine 1:837-839.
Bird et al. (1988) Single-chain antigen-binding proteins. Science 242:423-426.
Brennan et al. (1985) Preparation of bispecific antibodies by chemical recombination of monoclonal immunoglobulin G1 fragments. Science 229:81-83.
Bruhns & Jonsson (2015). Mouse and human FcR effector functions. Immunol Rev 268:25-51.
Burkholder et al. (2014) Tumor-induced perturbations of cytokines and immune cell networks. Biochim Biophys Acta 1845:182-201.
Burton & Barbas (1994) Human antibodies from combinatorial libraries. Adv Immunol 57:191-280.
Caballero et al. (2007) Ischemic vascular damage can be repaired by healthy, but not diabetic, endothelial progenitor cells. Diabetes 56:960-967.
Carter et al. (1992a) High level Escherichia coli expression and production of a bivalent humanized antibody fragment. Bio/Technology 10:163-167.
Carter et al. (1992b) Humanization of an anti-p185HER2 antibody for human cancer therapy. Proc Natl Acad Sci USA 89:4285.
Chakraborty et al. (2012) Robust and cost effective expansion of human regulatory T cells highly functional in a xenograft model of graft-versus-host disease. Haematologica 98(4):533-537.
Chiosea et al. (2015) Molecular characterization of apocrine salivary duct carcinoma. Am J Surg Pathol 39:744-752.
Choi et al. (2013) Gender-dimorphic regulation of skeletal muscle proteins in streptozotocin-induced diabetic rats. Cell Physiol Biochem 31:408-420.
Chothia & Lesk (1987) Canonical structures for the hypervariable regions of immunoglobulins. J Mol Biol 196:901-917.
Chung & Antoniades (1992) Expression of c-sis/platelet-derived growth factor B, insulin-like growth factor I, and transforming growth factor alpha messenger RNAs and their respective receptor messenger RNAs in primary human gastric carcinomas: in vivo studies with in situ hybridization and immunocytochemistry. Cancer Res 52:3453-3459.
Clauditz et al. (2013) Abundant expression of mTOR kinase in salivary gland tumors—potentials as therapy target? J Oral Pathol Med 42:769-773.
Cogle et al. (2014) Detailed analysis of bone marrow from patients with ischemic heart disease and left ventricular dysfunction. Circ Res 115:867-874.
Cole et al. (1985) in Monoclonal Antibodies and Cancer Therapy, Alan R. Liss, Inc., New York, New York, United States of America, pp. 77-96.
Cote et al. (1983) Generation of human monoclonal antibodies reactive with cellular antigens. Proc Natl Acad Sci USA 80:2026-2030.
Cunningham et al. (2004) Cetuximab monotherapy and cetuximab plus irinotecan in irinotecan-refractory metastatic colorectal cancer. N Engl J Med 351:337-345.
de Kruif et al. (1995) Selection and application of human single chain Fv antibody fragments from a semi-synthetic phage antibody display library with designed CDR3 regions. J Mol Biol 248:97-105.
Devereux et al. (1984) A comprehensive set of sequence analysis programs for the VAX. Nucl Acids Res 12:387-395.
Ellis et al. (2012) Generation of Induced Regulatory T Cells from Primary Human Naïve and Memory T Cells. J Vis Exp 62:3738.
Fadini et al. (2005) Circulating endothelial progenitor cells are reduced in peripheral vascular complications of type 2 diabetes mellitus. J Am Coll Cardiol 45:1449-1457.
Fortunato et al. (2013) Migratory activity of circulating progenitor cells and serum SDF-la predict adverse events in patients with myocardial infarction. Cardiovas Res 100:192-200.
Genaro (1985) Remington's Pharmaceutical Sciences, Mack Publishing Co., Easton, Pennsylvania, United States of America.
Grabert et al. (2006) Human T cells armed with Her2/neu bispecific antibodies divide, are cytotoxic, and secrete cytokines with repeated stimulation. Clin Cancer Res 12:569-576.
Green & Sambrook (2012) Molecular Cloning: A Laboratory Manual, Fourth Edition, Cold Spring Harbor Laboratory Press, Cold Spring Harbor, New York, United States of America.
Gross & Mienhofer (1981) The Peptides, Vol. 3, Academic Press, New York, New York, United States of America, pp. 3-88.
Gu et al. (1997) Construction and expression of mouse-human chimeric antibody SZ-51 specific for activated platelet P-selectin. Thromb Haemost 77(4):755-759.
Harlow & Lane (1988) Antibodies, A Laboratory Manual, Cold Spring Harbor Laboratory Publications, Cold Spring Harbor, New York, United States of America.
Heyman & Yang (2019) Chimeric Antigen Receptor T Cell Therapy for Solid Tumors: Current Status, Obstacles and Future Strategies. Cancers (Basel) 11:191.
Hofmann et al. (2005) Monitoring of bone marrow cell homing into the infracted human myocardium. Circulation 111:2198-2202.
Huse et al. (1989) Generation of a large combinatorial library of the immunoglobulin repertoire in phage lambda. Science 246:1275-1281.
Huston et al. (1988) Protein engineering of antibody binding sites: recovery of specific activity in an anti-digoxin single-chain Fv analogue produced in Escherichia coli. Proc Natl Acad Sci USA 85:5879-5883.
Jacoby & Nesto (1992) Acute myocardial infarction in the diabetic patient: pathophysiology, clinical course and prognosis. J Am Coll Cardiol 20:736-744.
Jarajapu & Grant (2010) The promise of cell-based therapies for diabetic complications: challenges and solutions. Circ Res 106:854-869.
Jones et al. (1986) Replacing the complementarity-determining regions in a human antibody with those from a mouse. Nature 321:522-525.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Kohler & Milstein (1975) Continuous cultures of fused cells secreting antibody of predefined specificity. Nature 256:495-497.
Kozbor & Roder (1983) The production of monoclonal antibodies from human lymphocytes. Immunology Today 4:72.
Lanza et al. (1999) Assessment of distribution of CD34 epitope classes in fresh and cryopreserved peripheral blood progenitor cells and acute myeloid leukemic blasts. Haematologica 84(11):969-977.
Lee & Margolin (2011) Cytokines in cancer immunotherapy. Cancers (Basel) 3:3856-3893.
Lee et al. (2007) Antibody Targeting of Stem Cells to Infarcted Myocardium. Stem Cells 25:712-717.
Lum & Sen (2001) Activated T cell and bispecific antibody immunotherapy for high-risk breast cancer. Bench to bedside. Acta Haematol. 105, 130-136 (2001).
Lum et al. (2015) Targeted T cell Therapy in Stage IV Breast Cancer: A Phase I Clinical Trial. Clin Cancer Res 21:2305-2314.
Lum et al. (2020a) Anti-CS1×Anti-CD3 Bispecific Antibody (BiAb)-Armed Anti-CD3 Activated T Cells (CS1-BATs) Kill CS1(+) Myeloma Cells and Release Type-1 Cytokines. Front Oncol 10:544.
Lum et al. (2020b) Clinical and immune responses to anti-CD3×anti-EGFR bispecific antibody armed activated T cells (EGFR BATs) in pancreatic cancer patients. OncoImmunology 9(1):1773201.
Luo et al. (2013) Diabetes increases mortality after myocardial infarction by oxidizing CaMKII. J Clin Invest 123:1262-1274.
Malecki et al. (2013) Recruitment and retention of human autologous CD34+CD117+CD133+ bone marrow stem cells to infarcted myocardium followed by directed vasculogenesis: novel strategy for cardiac regeneration. Mo Cell Ther 1:4.
Marks et al. (1991) By-passing immunization: human antibodies from V-gene libraries displayed on phage. J Mol Biol 222:581-597.
Matsuzaki et al. (2015) Direct tumor recognition by a human CD4(+) T cell subset potently mediates tumor growth inhibition and orchestrates anti-tumor immune responses. Sci Rep 5:14896.
Morimoto & Inouye (1992) Single-step purification of F(ab′)₂ fragments of mouse monoclonal antibodies (immunoglobulins G1) by hydrophobic interaction high performance liquid chromatography using TSKgel Phenyl-5PW. J Biochem Biophys Methods 24:107-117.
Morrison et al. (1984) Chimeric human antibody molecules: mouse antigen-binding domains with human constant region domains. Proc Natl Acad Sci USA. 81:6851-6855.
Neostem (2015) http://www.caladrius.com/wp-content/uploads/2015/05/NeoStem-Investor-Presentation.pdf.
Nesto & Zarich (1998) Acute myocardial infarction in diabetes mellitus: lessons learned from ACE inhibition. Circulation 97:12-15.
Neuberger et al. (1984) Recombinant antibodies possessing novel effector functions. Nature 312:604-608.
North et al. (2011) Native MAG-1 antibody almost destroys human breast cancer xenografts. Breast Cancer Res Treat 127(3):631-637.
Paul (1993) Fundamental Immunology, 3rd Ed., Raven Press, New York, New York, United States of America.
PCT International Patent Application Publication No. WO 92/02190.
Presta (1992) Antibody engineering. Curr Op Struct Biol 2:593-596.
Presta et al. (1993) Humanization of an antibody directed against IgE. J Immunol 1993 151:2623.
Qian et al. (2008) Development of new versions of anti-human CD34 monoclonal antibodies with potentially reduced immunogenicity. Biochem Biophys Res Commun 367(2):497-502.
Reusch et al. (2006) Anti-CD3×anti-EGFR Bispecific Antibody Redirects T Cell Cytolytic Activity to EGFR-Positive Cancers In Vitro and in an Animal Model. Clin Cancer Res 12:183-190.
Richel et al. (2000) Highly purified CD34+ cells isolated using magnetically activated cell selection provide rapid engraftment following high-dose chemotherapy in breast cancer patients. Bone Marrow Transplant 25:243-249.
Riechmann et al. (1988) Reshaping human antibodies for therapy. Nature 332:323-327.
Roche & Wang (2013) Sex differences in all-cause and cardiovascular mortality, hospitalization for individuals with and without diabetes, and patients with diabetes diagnosed early and late. Diabetes Care 36:2582-2590.
Roddie et al. (2019) Manufacturing chimeric antigen receptor T cells: issues and challenges. Cytotherapy 21(3):327-340.
Rubinstein et al. (1998) Anti-CD34+ Fabs generated against hematopoietic stem cells in HIV-derived combinatorial immunoglobulin library suggest antigen-selected autoantibodies. Mol Immunol 35(14-15):955-964.
Salomon et al. (1995) Epidermal growth factor-related peptides and their receptors in human malignancies. Crit Rev Oncol Hematol 19:183-232.
Saunders (2019) Conceptual Approaches to Modulating Antibody Effector Functions and Circulation Half-Life. Front Immunol 10:1296.
Schachinger et al. (2006a). Intracoronary bone marrow-derived progenitor cells in acute myocardial infarction. N Engl J Med 355:1210-1221.
Schachinger et al. (2006b) Improved clinical outcome after intracoronary administration of bone-marrow-derived progenitor cells in acute myocardial infarction: final 1-year results of the REPAIR-AMI trial. Eur Heart J 27:2775-2783.
Schmidts & Maus (2018) Making CART Cells a Solid Option for Solid Tumors. Front Immunol 9:2593.
Sen et al. (2001) Use of anti-CD3×anti-HER2/neu bispecific antibody for redirecting cytotoxicity of activated T cells toward HER2/neu+ tumors. J Hematother Stem Cell Res 10:247-260.
Shklovskaya et al. (2016) Tumour-specific CD4 T cells eradicate melanoma via indirect recognition of tumour-derived antigen. Immunol Cell Biol 94:593-603.
Showalter et al. (2017) Cytokines in immunogenic cell death: Applications for cancer immunotherapy. Cytokine 97:123-132.
Siegel et al. (2019) Cancer Statistics, 2019. CA Cancer J Clin 69(1):7-34.
Sims et al. (1993) A humanized CD18 antibody can block function without cell destruction. J Immunol 151:2296-2308.
Sokol & Luster (2015) The chemokine system in innate immunity. Cold Spring Harb Perspect Biol 7(5):a016303.
Strohl & Naso (2019) Bispecific T cell Redirection versus Chimeric Antigen Receptor (CAR)-T Cells as Approaches to Kill Cancer Cells. Antibodies (Basel) 8(3):41.
Strohl & Naso (2019) Bispecific T cell Redirection versus Chimeric Antigen Receptor (CAR)-T Cells as Approaches to Kill Cancer Cells. Antibodies (Basel) 8(3):41.
Subramanian et al. (2014) Stromal cell-derived factor 1 as a biomarker of heart failure and mortality risk. Thromb Vase Biol 34:2100-2105.
Takeda et al. (1985) Construction of chimaeric processed immunoglobulin genes containing mouse variable and human constant region sequences. Nature 314:452-454.
Tan & Yu (2007) Molecular mechanisms of erbB2-mediated breast cancer chemoresistance. Adv Exp Med Biol 608:119-129.
Thakur et al. (2013) Microenvironment generated during EGFR targeted killing of pancreatic tumor cells by ATC inhibits myeloid-derived suppressor cells through COX2 and PGE2 dependent pathway. J Transl Med 11:35.
Thakur et al. Immune T cells can transfer and boost anti-breast cancer immunity. OncoImmunology 7(12):e1500672.
Traverse et al. (2011) Effect of intracoronary delivery of autologous bone marrow mononuclear cells 2 to 3 weeks following acute myocardial infarction on left ventricular function. JAMA 306(19):2110.
Traverse et al. (2012) Effect of the use and timing of bone marrow mononuclear cell delivery on left ventricular function after acute myocardial infarction. JAMA 308(22):2380-2389.
Tuszynski et al. (1988) Thrombospondin promotes platelet aggregation. Blood 72:109-115.
U.S. Patent Application Publication Nos. 2002/0034765; 2003/0017534; 2003/0022244; 2003/0153043; 2004/0253645; 2006/0073137; 2018/0298087; 2018/0312588; 2018/0346564; 2019/0151448.
U.S. Pat. Nos. 4,816,567; 4,946,778; 5,001,065; 5,225,539; 5,482,856; 5,530,101; 5,585,089; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,479,284; 6,548,640; 6,632,927; 6,639,055; 6,677,436; 6,750,325; 6,797,492; 7,060,808; 7,906,625; 8,398,980; 8,436,150; 8,796,439; 10,253,111.
Vaishampayan et al. (2015) Phase I Study of Anti-CD3×Anti-Her2 Bispecific Antibody in Metastatic Castrate Resistant Prostate Cancer Patients. Prostate Cancer 2015:285193.
Verhoeyen et al. (1988) Reshaping human antibodies: grafting an antilysozyme activity. Science 239:1534-1536.
Vidarsson et al. (2014) IgG subclasses and allotypes: from structure to effector functions. Front Immunol 5:520.
Wagner et al. (2016) Phase I/II Trial of StemRegenin-1 Expanded Umbilical Cord Blood Hematopoietic Stem Cells Supports Testing as a Stand-Alone Graft. Cell Stem Cell 18:144-155.
Winter & Milstein (1991) Man-made antibodies. Nature 349:293-299.
Wright et al. (1992) Genetically engineered antibodies: progress and prospects. Critical Rev in Immunol 12(3,4):125-168.
Wu et al. (2020) Multiple signaling roles of CD3e and its application in CAR-T cell therapy. Cell 182:855-871.
Xing et al. (2009) Optimal time for human umbilical cord blood cell transplantation in rats with myocardial infarction. Chin Med J 122(23):2833-2839.
Yan et al. (2015) HER2 expression status in diverse cancers: review of results from 37,992 patients. Cancer Metastasis Rev 34:157-164.
Yankelevich et al. (2019) Phase I study of OKT3×hu3F8 bispecific antibody (GD2Bi) armed T cells (GD2BATs) in GD2-positive tumors. Journal of Clinical Oncology 37(15 Suppl):Abstract 2533.
Yano et al. (2014) Ipilimumab augments antitumor activity of bispecific antibody-armed T cells. Journal of translational medicine 12:191.
Yasui et al. (1988) Expression of epidermal growth factor receptor in human gastric and colonic carcinomas. Cancer Res 48:137-141.
Yu et al. (2015) Immuno-modification of enhancing stem cells targeting for myocardial repair. J Cell Mol Med 19(7):1483-1491.
Zhang et al. (2004) Both cell fusion and transdifferentiation account for the transformation of human peripheral blood CD34-positive cells into cardiomyocytes in vivo. Circulation 110:3803-3807.
Zhang et al. (2020) Engineering better chimeric antigen receptor T cells. Exp Hematol Oncol 9:34.
Zhao et al. (2008) Targeting human CD34+ hematopoietic stem cells with anti-CD45×anti-myosin light-chain bispecific antibody preserves cardiac function in myocardial infarction. J Appl Physiol 104:1793-1800.
Zimmet et al. (2012) Short- and long-term outcomes of intracoronary and endogenously mobilized bone marrow stem cells in the treatment of ST-segment elevation myocardial infarction: a meta-analysis of randomized control trials. Eur J Heart Fail 14:91-105.
Zitron et al. (2013) Targeting and killing of glioblastoma with activated T cells armed with bispecific antibodies. BMC Cancer 13:83.

It will be understood that various details of the presently disclosed subject matter can be changed without departing from the scope of the presently disclosed subject matter. Furthermore, the foregoing description is for the purpose of illustration only, and not for the purpose of limitation.

1. A tetravalent bispecific antibody (T-BiAb) comprising a first binding moiety and a second binding moiety, wherein the first binding moiety is a single chain variable fragment (scFv) and the second binding moiety is a monoclonal antibody, and further wherein the variable light (V_{L}) and variable heavy (V_{H}) chains of the first binding moiety are directly linked as a single chain to the second binding moiety at the N-terminus or the C-terminus of the light chain or the heavy chain sequence of the second binding moiety.

2. The T-BiAb of claim 1, wherein the variable light (V_{L}) and variable heavy (V_{H}) chains of the first binding moiety are linked to each other as a single polypeptide chain via a peptide linker.

3. The T-BiAb of claim 1, wherein the peptide linker comprises, consists essentially of, or consists of the amino acid sequence GGGGS (SEQ ID NO: 6), optionally wherein the peptide linker comprises, consists essentially of, or consists of a concatemer of 3-6 copies of the amino acid sequence GGGGS (SEQ ID NO: 6).

4. The T-BiAb of claim 1, wherein at least one of the copies of the amino acid sequence GGGGS (SEQ ID NO: 6) includes an amino acid substitution to GGGTS (SEQ ID NO: 7), optionally wherein the peptide linker comprises, consists essentially of, or consists of the amino acid sequence GGGGSGGGGSGGGTSGGGGSGGGGS (SEQ ID NO: 10) or GGGGSGGGGSGGGTSGGGGSGGGGSGGGGS (SEQ ID NO: 13).

5. The T-BiAb of claim 1, wherein the variable light (V_{L}) and variable heavy (V_{H}) chains of the scFv are linked to each other in a configuration selected from the group consisting of V_{L}-(G4S)ₓ-V_{H} and V_{H}-(G4S)ₓ-V_{L}, wherein G4S is the amino acid sequence GGGGS (SEQ ID NO: 6) or a threonine-containing variant thereof and x is 3-6.

6. The T-BiAb of claim 1, wherein the scFv binds to a CD3 polypeptide, optionally wherein the scFv is an scFv of an OKT3 monoclonal antibody, and further optionally wherein the second binding moiety binds to a tumor-associated antigen.

7. (canceled)

8. The T-BiAb of claim 1, wherein the tumor-associated antigen is a polypeptide selected from the group consisting of an ERBB family member polypeptide, optionally an epidermal growth factor receptor (EGFR/ERBB1) polypeptide, a HER2/ERBB2 polypeptide, a HER3/ERBB3 polypeptide, a HER4/ERBB4 polypeptide, a disialoganglioside 2 (GD2) polypeptide, a MAG-1 polypeptide, a CD19 polypeptide, a CD20 polypeptide, a CD22 polypeptide, a CD30 polypeptide, a CD33 polypeptide, a CD34 polypeptide, a CS1/SLAMF7 polypeptide, a B cell maturation antigen (BCMA) polypeptide, a CD38 polypeptide, and a CD123 polypeptide.

9. A T cell armed with a T-BiAb of claim 1.

10. A method for treating a tumor and/or a cancer, the method comprising contacting the tumor and/or the cancer with an effective amount of a composition comprising at least one T-BiAb of claim 1.

11. The method of claim 10, wherein the tumor and/or the cancer is selected from the group consisting of a breast tumor and/or cancer, a pancreatic tumor and/or cancer, a prostate tumor and/or cancer, or a glioblastoma.

12. A method for treating diabetes, the method comprising contacting a β-cell in a subject with an effective amount of a composition comprising one or more T-BiAbs, wherein each T-BiAb comprises a first binding moiety that binds to CD34 or CD45 and a second binding moiety that binds to a myosin light chain (MLC) polypeptide, wherein the first binding moiety is a single chain variable fragment (scFv) and the second binding moiety is a monoclonal antibody, and further wherein the variable light (V_{L}) and variable heavy (V_{H}) chains of the first binding moiety are directly linked as a single chain to the second binding moiety at the N-terminus or the C-terminus of the light chain or the heavy chain sequence of the second binding moiety.

13. The method of claim 12, wherein the T-BiAb is bound to a stem cell.

14. (canceled)

15. A method for activating a T cell, the method comprising, consisting essentially of, or consisting of contacting a T cell with a tetravalent bispecific antibody (T-BiAb) in an amount sufficient to activate the T cell, wherein the T-BiAb comprises, consists of, or consisting of a first binding moiety and a second binding moiety, and further wherein:
(i) the first binding moiety is a single chain variable fragment (scFv) comprising a variable light (VL) chain and a variable heavy (VH) chain;
(ii) the second binding moiety is a monoclonal antibody comprising a light chain and a heavy chain; and
(iii) the variable light (VL) chain and the variable heavy (VH) chain of the first binding moiety are directly linked as a single chain to the second binding moiety at the N-terminus or the C-terminus of the light chain or the heavy chain sequence of the second binding moiety,
to thereby generate an activated T cell.

16. The method of claim 15, wherein the scFv binds to an CD3 polypeptide, optionally wherein the scFv is an scFv of an OKT3 monoclonal antibody.

17. The method of claim 15, wherein the scFv binds to a CD3 polypeptide and the second binding moiety binds to a tumor-associated antigen (TAA).

18. The method of claim 17, wherein the TAA is a polypeptide selected from the group consisting of an ERBB family member polypeptide, optionally an epidermal growth factor receptor (EGFR/ERBB1) polypeptide, a HER2/ERBB2 polypeptide, a HER3/ERBB3 polypeptide, a HER4/ERBB4 polypeptide, a disialoganglioside 2 (GD2) polypeptide, a MAG-1 polypeptide, a CD19 polypeptide, a CD20 polypeptide, a CD22 polypeptide, a CD30 polypeptide, a CD33 polypeptide, a CD34 polypeptide, a CS1/SLAMF7 polypeptide, a B cell maturation antigen (BCMA) polypeptide, a CD38 polypeptide, and a CD123 polypeptide.

19. The method of claim 15, wherein the T cell is derived from a peripheral blood mononuclear cell (PBMC) or a tumor infiltrating T cell.

20. The method of claim 15, wherein the T cell is a modified T cell that expresses a chimeric antigen receptor (CAR), optionally wherein the CAR is encoded by a transgene.

21. The method of claim 15, further comprising converting the activated T cell to a CD4+/CD25+/FoxP3+ T regulatory (Treg) cell or a CAR-T cell.

22-33. (canceled)

34. The armed T cell of claim 9, further comprising a pharmaceutically acceptable carrier, excipient, and/or diluent, optionally wherein the pharmaceutically acceptable carrier, excipient, and/or diluent is pharmaceutically acceptable for use in a human.

35. (canceled)
